SEQUENCE LISTING (42 sequences)

SEQ ID NO: 1 - protein, 8 aa, Artificial Sequence
  VH-CDR1
GYTFTSYW

SEQ ID NO: 2 - protein, 8 aa, Artificial Sequence
  VH-CDR2
IFPGTGTT

SEQ ID NO: 3 - protein, 6 aa, Artificial Sequence
  VH-CDR3
SRDFDV

SEQ ID NO: 4 - protein, 11 aa, Artificial Sequence
  VL-CDR1
QSLLDIDGKTY

SEQ ID NO: 5 - protein, 9 aa, Artificial Sequence
  VL-CDR3
WQGTHLPRT

SEQ ID NO: 6 - protein, 112 aa, Artificial Sequence
  VH
  SITE 39: X is Q or R
  SITE 98: X is R or G
  SITE 107: X is T or A
  SITE 108: X is S or T
QVQLQQSGAELVKPGTSVKLSCKTSGYTFTSYWIQWIKXRPGQGLGWIGEIFPGTGTTSYHEKFKGKATLTIDTSSSTAYLQLSNLTSEDSAVYFCSXDFDVWGAGXXVTVS

SEQ ID NO: 7 - protein, 112 aa, Artificial Sequence
  VL
  SITE 9: X is P or L
  SITE 48: X is S or F
  SITE 81: X is S or Absent
DVVMTQTPXTLSVTIGQPASISCKSSQSLLDIDGKTYLNWLLQRPGQXPKRLIYLVSKLDSGVPDRVTGSGSGTDFTLKIXRVEAEDLGVYYCWQGTHLPRTFGGGTNLEIK

SEQ ID NO: 8 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSRDFDVWGAGTTVTVSS

SEQ ID NO: 9 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSRDFDVWGAGATVTVSS

SEQ ID NO: 10 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSRDFDVWGAGTSVTVSS

SEQ ID NO: 11 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSRDFDVWGAGTTVTVSS

SEQ ID NO: 12 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSGDFDVWGAGASVTVSS

SEQ ID NO: 13 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSGDFDVWGAGATVTVSS

SEQ ID NO: 14 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSGDFDVWGAGTSVTVSS

SEQ ID NO: 15 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSGDFDVWGAGTTVTVSS

SEQ ID NO: 16 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSRDFDVWGAGASVTVSS

SEQ ID NO: 17 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSRDFDVWGAGATVTVSS

SEQ ID NO: 18 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSRDFDVWGAGTSVTVSS

SEQ ID NO: 19 - protein, 113 aa, Artificial Sequence
  VH
QVQLQQSGAELVKPGTSVKLSCKTSGYTFTSYWIQWIKQRPGQGLGWIGEIFPGTGTTSYHEKFKGKATLTIDTSSSTAYLQLSNLTSEDSAVYFCSRDFDVWGAGASVTVSS

SEQ ID NO: 20 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSGDFDVWGAGASVTVSS

SEQ ID NO: 21 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSGDFDVWGAGATVTVSS

SEQ ID NO: 22 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSGDFDVWGAGTSVTVSS

SEQ ID NO: 23 - protein, 113 aa, Artificial Sequence
  VH
QVQLQQSGAELVKPGTSVKLSCKTSGYTFTSYWIQWIKRRPGQGLGWIGEIFPGTGTTSYHEKFKGKATLTIDTSSSTAYLQLSNLTSEDSAVYFCSGDFDVWGAGTTVTVSS

SEQ ID NO: 24 - protein, 112 aa, Artificial Sequence
  VL
DVVMTQTPLTLSVTIGQPASISCKSSQSLLDIDGKTYLNWLLQRPGQSPKRLIYLVSKLDSGVPDRVTGSGSGTDFTLKISRVEAEDLGVYYCWQGTHLPRTFGGGTNLEIK

SEQ ID NO: 25 - protein, 111 aa, Artificial Sequence
  VL
DVVMTQTPPTLSVTIGQPASISCKSSQSLLDIDGKTYLNWLLQRPGQSPKRLIYLVSKLDSGVPDRVTGSGSGTDFTLKIRVEAEDLGVYYCWQGTHLPRTFGGGTNLEIK

SEQ ID NO: 26 - protein, 112 aa, Artificial Sequence
  VL
DVVMTQTPPTLSVTIGQPASISCKSSQSLLDIDGKTYLNWLLQRPGQFPKRLIYLVSKLDSGVPDRVTGSGSGTDFTLKISRVEAEDLGVYYCWQGTHLPRTFGGGTNLEIK

SEQ ID NO: 27 - protein, 111 aa, Artificial Sequence
  VL
DVVMTQTPPTLSVTIGQPASISCKSSQSLLDIDGKTYLNWLLQRPGQFPKRLIYLVSKLDSGVPDRVTGSGSGTDFTLKIRVEAEDLGVYYCWQGTHLPRTFGGGTNLEIK

SEQ ID NO: 28 - protein, 112 aa, Artificial Sequence
  VL
DVVMTQTPPTLSVTIGQPASISCKSSQSLLDIDGKTYLNWLLQRPGQSPKRLIYLVSKLDSGVPDRVTGSGSGTDFTLKISRVEAEDLGVYYCWQGTHLPRTFGGGTNLEIK

SEQ ID NO: 29 - protein, 111 aa, Artificial Sequence
  VL
DVVMTQTPLTLSVTIGQPASISCKSSQSLLDIDGKTYLNWLLQRPGQSPKRLIYLVSKLDSGVPDRVTGSGSGTDFTLKIRVEAEDLGVYYCWQGTHLPRTFGGGTNLEIK

SEQ ID NO: 30 - protein, 112 aa, Artificial Sequence
  VL
DVVMTQTPLTLSVTIGQPASISCKSSQSLLDIDGKTYLNWLLQRPGQFPKRLIYLVSKLDSGVPDRVTGSGSGTDFTLKISRVEAEDLGVYYCWQGTHLPRTFGGGTNLEIK

SEQ ID NO: 31 - protein, 111 aa, Artificial Sequence
  VL
DVVMTQTPLTLSVTIGQPASISCKSSQSLLDIDGKTYLNWLLQRPGQFPKRLIYLVSKLDSGVPDRVTGSGSGTDFTLKIRVEAEDLGVYYCWQGTHLPRTFGGGTNLEIK

SEQ ID NO: 32 - DNA, 340 bp, Artificial Sequence
  VH
caggtccagctgcagcagtctggagctgaactggtgaagcctgggacttcagtgaaactgtcctgcaagacttctggctacaccttcaccagctactggattcagtggataaaacagaggcctggacagggccttgggtggattggagagatatttcctggaactggcacgacttcctaccatgagaaattcaagggcaaggccacactgactatagacacatcctccagcacagcctacttgcagctcagcaacctgacctctgaagactctgctgtctatttctgttcaagagacttcgatgtctggggcgcaggcaccactgtcaccgtctcctcaa

SEQ ID NO: 33 - DNA, 337 bp, Artificial Sequence
  VL
gatgttgtgatgacccagactccactcactttgtcggttaccattgggcaaccagcctccatctcttgcaagtcaagtcagagcctcttagatattgatggaaagacatatttgaattggttgttacagaggccaggccagtctccaaagcgcctaatctatctggtgtctaaactggactctggagtccctgacagggtcactggcagtggatcagggacagatttcacactgaaaatcagcagagtggaggctgaggatttgggagtttattattgttggcaaggtacacatcttcctcggacgttcggtggaggcaccaacctggaaatcaaac

SEQ ID NO: 34 - protein, 879 aa, Homo sapiens
  CD9P-1
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 35 - protein, 13 aa, Artificial Sequence
  peptide
GNYYCSVTPWVKS

SEQ ID NO: 36 - protein, 17 aa, Artificial Sequence
  peptide
IHSKPVFITVKMDVLNA

SEQ ID NO: 37 - DNA, 19 bp, Artificial Sequence
  hCD9P1-F
caggagctggcacaaagtg

SEQ ID NO: 38 - DNA, 21 bp, Artificial Sequence
  hCD9P1-R
ccttggaagcattcaggtaca

SEQ ID NO: 39 - DNA, 20 bp, Artificial Sequence
  hGAPDH-F
agctcactggcatggccttc

SEQ ID NO: 40 - DNA, 20 bp, Artificial Sequence
  hGAPDH-R
gaggtccaccaccctgttgc

SEQ ID NO: 41 - DNA, 24 bp, Artificial Sequence
  mGAPDH-F
ggccttgactgtgccgttgaattt

SEQ ID NO: 42 - DNA, 24 bp, Artificial Sequence
  mGAPDH-R
ggccttgactgtgccgttgaattt

(19) United States
(12) Patent Application Publication
(10) Pub. No.: US 2020/0172618 A1
(43) Pub. Date: Jun. 4, 2020

(54) CD9P-1-TARGETING ANTIBODY AND USES THEREOF

(71) Applicant: Gene Signal International SA, Lausanne (CH)

(72) Inventor: Sylvie Colin, Cachan (FR)

(73) Assignee: Gene Signal International SA, Lausanne (CH)

(21) Appl. No.: 16/634,407

(22) PCT Filed: Jul. 27, 2018
(86) PCT No.: PCT/EP2018/070469

§ 371 Date: Jan. 27, 2020

Related U.S. Application Data
(60) Provisional application No. 62/538,106, filed on Jul. 28, 2017.

(30) Foreign Application Priority Data
Jul. 28, 2017 (EP) 17306017.9

(51) Int. Cl. C07K 16/28; A61P 35/00

(52) U.S. Cl. CPC C07K 16/2803; C07K 2317/76; A61P 35/00

(57) ABSTRACT

The present disclosure relates to an isolated protein that inhibits the CD9P-1 pathway, preferably that inhibits the CD9P-1stabilin-1 pathway and/or the CD9P-1TRAF-2 pathway, in particular to an isolated antibody against human CD9P-1, and to the use thereof in therapeutic or diagnostic methods.

### FIELD OF INVENTION

The present invention relates to proteins that target human Cluster of Differentiation 9 partner 1 (CD9P-1), in particular to novel antibodies against CD9P-1, and to the uses thereof in treatment and diagnostic methods.

### BACKGROUND OF INVENTION

The unstoppable growth and spread of chemically-resistant tumor metastasis is one of the leading cause of death from cancer. Tumor metastasis can arise from clonal division and originate from many different progenitor cells. Moreover, metastatic cells exhibit a higher rate of spontaneous mutation and a defective DNA repair machinery compared with non-neoplastic cells. All of this allow explaining the existence of patients non-responding to conventional therapeutic drugs due to the wide range of sensitivities of metastases to the same chemotherapy. Therefore, cancer immunotherapy, which manipulates the host immune response towards cancer cells, has become one of the cornerstones of cancer care, alongside with surgery, cytotoxic treatment and radiotherapy.

The recent clinical results using immune checkpoint blockade with anti-CTL4 and anti-PDL1/anti-PD1 monoclonal antibodies, have clearly established immunotherapy as a very promising approach for the treatment of cancer. Checkpoint inhibitors (CPI, that may also be referred to as Immune checkpoint inhibitors or ICI) target normal immune cells in order to stimulate the antitumor response. They work by blocking interactions between inhibitory receptors expressed on T cells and their ligands. Their efficacy was demonstrated in multiple types of cancer. However, only a fraction of patients respond to these therapies and new immunotherapeutic targets are necessary.

Targeting of tumor-associated macrophages is also considered as a promising immunotherapeutic strategy. Indeed, tumoricidal-macrophages, a major constituent of tumor stroma in solid tumors, can recognize, uptake and degrade neoplastic cells while leaving metastatic cells uninjured, making it an outstanding target for the prevention and treatment of tumorigenesis. Therefore, the following strategies are currently tested in clinical trials in order to develop novel anti-cancer treatments: inhibition of macrophage recruitment into the tumor environment (CSF-1R inhibitors), development of tumoricidal effector (BCG), induction of a depletion of M2 TAMs or “re-education” of them as anti-tumor effectors, “M1-like” mode (anti-CD40); and development of tumor-targeting monoclonal antibodies that elicit phagocytosis and intracellular destruction of cancer cells (anti-CD47).

Strategies which co-opts both nonspecific innate immunity and antigen-specific, memory-promoting adaptive immunity for tumor destruction are the most promising cancer therapies. Therapeutic combinations which induce antitumor responses driven by elements of innate immunity, including, for example, natural killer (NK), macrophages, granulocytes and dendritic cells that induce cell death by activating apoptosis, phagocytosis, and costimulatory pathway and/or elements of adaptive immunity, in particular lymphocytes (B cells and T cells) involved in the humoral immune response (B cells) or in cell-mediated immune responses (T cells). Effective activation of T cells requires engagement of two separate T-cell receptors. The antigen-specific T-cell receptor (TCR) binds foreign peptide antigen-MHC complexes, and the CD28 receptor binds to the B7 (CD80/CD86) costimulatory molecules expressed on the surface of antigen-presenting cells (APC) which may be monocytes, macrophages, dendritic cells or B lymphocytes.

Tetraspanin proteins make various contributions to tumorigenesis, notably by supporting tumor growth, spread and angiogenesis. CD9 is the most studied tetraspanin: it is known to function in multiple cell events, including membrane fusion, differentiation and cell motility and seems to have a key role in metastasis. Clinical observations suggest that downregulation of CD9 is associated with the progression of solid tumors. Tetraspanins form a family of proteins with four transmembrane domains delineating two extracellular domains of unequal size, which are involved in numerous physiological processes including angiogenesis, cell migration, cell-cell contact and fusion. The function of tetraspanins is thought to be related to their ability to interact with one another and with various other surface proteins, forming a network of molecular interactions referred to as the tetraspanin web. Through their partner protein interactions, tetraspanins are also implicated in the immune response against cancer. Tetraspanins participate in antigen recognition and presentation by antigen presenting cells (APCs) through the organization of pattern-recognition receptors (PRRs) and their downstream-induced signaling, as well as the regulation of MHC-II-peptide trafficking. CD9 regulates MHC-II trafficking in monocyte-derived dendritic cells. CD81 modulates the immune suppression in cancer and metastasis.

CD9 partner 1 (also known as “CD9P-1”, “FPRP” or “EWI-F”) that belongs to the Immunoglobulin superfamily (IgSF), associates with CD9, CD81 and CD151 and localizes therefore within the tetraspanins web. CD9P-1 is a glycosylated, type 1 integral membrane protein, associated with lipid microdomains, and overexpressed by a large set of cancer cell lines. CD9P-1 gene expression has been shown to correlate with the metastatic status of cancer, and it is suggested that CD9P-1 may be a major contributor to the loss of CD9 expression in solid tumors. In particular, GS-168AT2, a truncated form of CD9P-1, potently inhibits tumor-induced angiogenesis in nude mice, thereby limiting in vivo tumor growth. Moreover, transfection of human embryonic kidney cells (HEK 293) with CD9P-1 coding vector increased significantly cell migration, i.e., most probably tumor-cell invasion. Thus, there is a pressing need to develop specific inhibitors of CD9P-1 for cancer therapies.

The present inventors have developed monoclonal antibodies (hereafter 9bF4 and 10bB1) specifically targeting the CD9P-1 protein. Surprisingly, these antibodies actually possess on their own an effective anticancer activity, in particular through the induction of cancer cells apoptosis.

Preclinical and clinical studies have shown that macrophages can be a major constituent of tumor in various cancers, entailing a poor prognosis by promoting angiogenesis and metastasis. These tumor-associated macrophages (TAMs) are thought to express an M2 phenotype. Surprisingly, the antibodies of the present invention were shown to trigger M2 to M1 macrophage repolarization, thereby limiting the deleterious anti-inflammatory and protumor effects of M2 state TAMs. Further, the antibodies of the present invention were advantageously demonstrated to induce cancer cell apoptosis through monocyte and lymphocyte proliferation/activation. 9bF4 and 10bB1 antibodies are therefore constituting unexpected and advantageous alternatives to the known-to-date cancer treatments, since they are, for instance, capable to induce the expression of TNF-alpha and IFN-gamma, two major tumoricidal cytokines and key effectors of immune response, without the drawbacks of its recombinant counterparts (such as, for example, difficulty to produce in large quantities, loss of biological activity upon long-term storage, administration to patients resulting in side effects, etc.).

The present invention thus relates to novel isolated proteins targeting human CD9P-1, in particular to novel antibodies directed to CD9P-1, and to the therapeutic and diagnostic thereof.

### SUMMARY

The present invention relates to an isolated protein that inhibits the CD9P-1 pathway, preferably that inhibits the CD9P-1stabilin-1 pathway and/or the CD9P-1TRAF-2 pathway.

In one embodiment, said protein induces an internalization and/or degradation of CD9P-1, and preferably further induces an internalization and/or degradation of Stabilin-1 and/or a degradation of TRAF-2.

In one embodiment, the protein of the invention binds to CD9P-1, preferably said protein is an antibody molecule selected from the group consisting of a whole antibody, a humanized antibody, a single chain antibody, a dimeric single chain antibody, a Fv, a Fab, a F(ab)′2, a defucosylated antibody, a bi-specific antibody, a diabody, a triabody, a tetrabody, an antibody fragment selected from the group consisting of a unibody, a domain antibody, and a nanobody or an antibody mimetic selected from the group consisting of an affibody, an affilin, an affitin, an adnectin, an atrimer, an evasin, a DARPin, an anticalin, an avimer, a fynomer, a versabody and a duocalin.

In one embodiment, the protein of the invention binds to a conformational epitope comprising:
at least one amino acid residue from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34), and
at least one amino acid residue from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34), and
at least one amino acid residue from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, the variable region of the heavy chain comprises at least one of the following CDRs:

| | | |
|---|---|---|
| VH-CDR1: | GYTFTSYW; | (SEQ ID NO: 1) |
| VH-CDR2: | IFPGTGTT; | (SEQ ID NO: 2) |
| and | | |
| VH-CDR3: | SRDFDV, | (SEQ ID NO: 3) |

or any CDR having an amino acid sequence that shares at least 60% of identity with SEQ ID NO: 1-3, and/or

the variable region of the light chain comprises at least one of the following CDRs:

| | | |
|---|---|---|
| VL-CDR1: | QSLLDIDGKTY; | (SEQ ID NO: 4) |
| VL-CDR2: | LVS; | |
| and | | |
| VL-CDR3: | WQGTHLPRT, | (SEQ ID NO: 5) |

or any CDR having an amino acid sequence that shares at least 60% of identity with SEQ ID NO: 4, LVS and SEQ ID NO: 5.

In one embodiment, the variable region of the heavy chain comprises at least one of the CDRs as defined hereinabove and the variable region of the light chain comprises at least one of the CDRs as defined hereinabove.

In one embodiment, the variable region of the heavy chain comprises the following CDRs: GYTFTSYW (SEQ ID NO: 1), IFPGTGTT (SEQ ID NO: 2) and SRDFDV (SEQ ID NO: 3) and the variable region of the light chain comprises the following CDRs: QSLLDIDGKTY (SEQ ID NO: 4), LVS and WQGTHLPRT (SEQ ID NO: 5) or any CDR having an amino acid sequence that shares at least 60% of identity with said SEQ ID NO: 1-5 and LVS.

In one embodiment, the amino acid sequence of the heavy chain variable region is SEQ ID NO: 6 wherein X₁ is Q or R, X₂ is R or G, X₃ is T or A, X₄ is S or T and the amino acid sequence of the light variable region is SEQ ID NO: 7 wherein X₅ is P or L, X₆ is S or F, and X₇ is S or absent, or any sequence having an amino acid sequence that shares at least 60% of identity with said SEQ ID NO: 6-7.

In one embodiment, in SEQ ID NO: 6, X₁ is R, X₂ is R,X₃ is T and X₄ is T (SEQ ID NO: 8), and/or in SEQ ID NO: 7, X₅ is L, X₆ is S and X₇ is S (SEQ ID NO: 24).

In one embodiment, in SEQ ID NO: 6, X₁ is Q, X₂ is R,X₃ is T and X₄ is T (SEQ ID NO: 11), and/or in SEQ ID NO: 7, X₅ is L,X₆ is S and X₇ is S (SEQ ID NO: 24).

The present invention further relates to a composition comprising a protein as defined hereinabove.

The present invention further relates to an isolated protein as described hereinabove, for treating a cancer.

The present invention further relates to the use of the protein as described hereinabove for detecting CD9P-1 in a biological sample.

The present invention further relates to an in vitro diagnostic or prognostic assay for determining the presence of CD9P-1 in a biological sample, preferably the 135 kDa glycosylated transmembrane form of CD9P-1, using a protein as described hereinabove.

In one embodiment, the assay is a sandwich ELISA using an antibody as described hereinabove as coating antibody.

The present invention further relates to a kit comprising at least one antibody against human CD9P-1 as described hereinabove, preferably the antibody characterized by the 6 CDRs sequences shown hereinabove, and optionally a revealing antibody.

The present invention further relates to an expression vector comprising at least one of SEQ ID NO: 32 and SEQ ID NO: 33 or any sequence having a nucleic acid sequence that shares at least 60% of identity with said SEQ ID NO: 32-33.

The present invention further relates to hybridoma cell lines producing an antibody against human CD9P-1 registered under CNCM1-5213 and CNCM1-5214.

The present invention further relates to a method for inducing apoptosis of cancer cells in a subject in need thereof, comprising administering to the subject an effective amount of the protein as described herein.

The present invention further relates to a method for inducing M2 macrophages repolarization in M1 macrophages in a subject in need thereof, comprising administering to the subject an effective amount of the protein as described herein.

The present invention further relates to a method for inducing an immune response and/or an inflammatory response in a subject in need thereof, comprising administering to the subject an effective amount of the protein as described herein.

DEFINITIONS

In the present invention, the following terms have the following meanings:

CD9P-1 is a cell surface protein with immunoglobulin domains and a major component of the tetraspanin web associating with tetraspanin protein CD9. The complete amino acid sequence of the human CD9P-1 protein (SEQ ID NO: 34) (Accession number NP_065173.2) is:

MGRLASRPLLLALLSLALCRG
(signal peptide)
RVVRVPTATLVRVVGTELVIPCNVSDYDGPSEQNFDWSFSSLGSSFVELA
STWEVGFPAQLYQERLQRGEILLRRTANDAVELHIKNVQPSDQGHYKCST
PSTDATVQGNYEDTVQVKVLADSLHVGPSARPPPSLSLREGEPFELRCTA
ASASPLHTHLALLWEVHRGPARRSVLALTHEGRFHPGLGYEQRYHSGDVR
LDTVGSDAYRLSVSRALSADQGSYRCIVSEWIAEQGNWQEIQEKAVEVAT
VVIQPSVLRAAVPKNVSVAEGKELDLTCNITTDRADDVRPEVTWSFSRMP
DSTLPGSRVLARLDRDSLVHSSPHVALSHVDARSYHLLVRDVSKENSGYY
YCHVSLWAPGHNRSWHKVAEAVSSPAGVGVTWLEPDYQVYLNASKVPGFA
DDPTELACRVVDTKSGEANVRFTVSWYYRMNRRSDNVVTSELLAVMDGDW
TLKYGERSKQRAQDGDFIFSKEHTDTFNFRIQRTTEEDRGNYYCVVSAWT
KQRNNSWVKSKDVFSKPVNIFWALEDSVLVVKARQPKPFFAAGNTFEMTC
KVSSKNIKSPRYSVLIMAEKPVGDLSSPNETKYIISLDQDSVVKLENWTD
ASRVDGVVLEKVQEDEFRYRMYQTQVSDAGLYRCMVTAWSPVRGSLWREA
ATSLSNPIEIDFQTSGPIFNASVHSDTPSVIRGDLIKLFCIITVEGAALD
PDDMAFDVSWFAVHSFGLDKAPVLLSSLDRKGIVTTSRRDWKSDLSLERV
SVLEFLLQVHGSEDQDFGNYYCSVTPWVKSPTGSWQKEAEIHSKPVFITV
KMDVLNAFKYP
(extracellular domain)
LLIGVGLSTVIGLLSCLIGYCSSHWCCKKEVQETRRERRRLMSMEMD
(transmembrane and intracellular domains)

The CD9P-1 protein is subject to post-translational modifications, including in particular glycosylation, i.e., the attachment of sugar moieties to amino acid residues. Glycosylation is critical for a wide range of biological processes, including allowing glycoproteins to act as ligands for receptors on the cell surface to mediate cell attachment or stimulate signal transduction pathways. Hence, the 135 kDa form of CD9P-1 more likely corresponds to a glycosylated transmembrane protein.

The term “about” preceding a figure means plus or less 10% of the value of said figure.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses of the antibody, and may include enzymes, hormones, and other proteinaceous or non-proteinaceous components. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity as shown by SDS-PAGE under reducing or non-reducing conditions and using Coomassie blue or, preferably, silver staining. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. The L chain from any vertebrate species can be assigned to one of two clearly distinct types, called kappa ([kappa]) and lambda ([lambda]), based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and. IgM, having heavy chains designated alpha ([alpha]), delta ([delta]), epsilon ([epsilon]), gamma ([gamma]) and mu ([mu]), respectively. The [gamma] and [alpha] classes are further divided into subclasses on the basis of relatively minor differences in CH sequence and function, e.g., humans express the following subclasses: IgG1, IgG2, IgG3, IgG4, IgA1, and IgA2. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the [alpha] and [gamma] chains and four CH domains for [mu] and [epsilon] isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. An IgM antibody consists of five of the basic heterotetramer units along with an additional polypeptide called a J chain, and therefore, contains ten antigen binding sites, while secreted IgA antibodies can polymerize to form polyvalent assemblages comprising 2-5 of the basic 4-chain units along with J chain. In the case of IgGs, the 4-chain unit is generally about 150,000 Daltons. For the structure and properties of the different classes of antibodies, see, e.g., Basic and Clinical Immunology, 8th edition, Daniel P. Stites, Abba I. Ten and Tristram G. Parslow (eds.), Appleton & Lange, Norwalk, Conn., 1994, page 71, and Chapter 6.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a [beta]-sheet configuration, connected by three hypervariable regions, which form loops connecting, and in some cases forming part of, the [beta]-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), complement-dependent cytotoxicity (CDC), or antibody-dependent cell phagocytosis (ADCP).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the VL, and around about 31-35 (H1), 50-65 (H2) and 95-102 (H3) in the VH when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the VL, and 26-32 (H1), 52-56 (H2) and 95-15 101 (H3) in the VH when numbered in accordance with the Chothia numbering system; Chothia and Lesk, J. Mot. Biol. 196:901-917 (1987)); and/or those residues from a “hypervariable loop”/CDR (e.g., residues 27-38 (L1), 56-65 (L2) and 105-120 (L3) in the VL, and 27-38 (H1), 56-65 (H2) and 105-120 (H3) in the VH when numbered in accordance with the IMGT numbering system; Lefranc, M. P. et al., Nucl. Acids Res. 27:209-212 (1999), Ruiz, M. et al., Nucl. Acids Res. 28:219-221 (2000)). Optionally the antibody has symmetrical insertions at one or more of the following points 28, 36 (L1), 63, 74-75 (L2) and 123 (L3) in the VL, and 28, 36 (H1), 63, 74-75 (H2) and 123 (H3) in the VH when numbered in accordance with AHo (Honneger, A. and Plunkthun, A. J. Mot. Biol. 309:657-670 (2001)).

The term “monoclonal antibody” as used herein refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). The present invention provides variable domain antigen-binding sequences derived from human antibodies.

Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc.).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al., (1994) Nature Genet. 7:13; Lonberg et 5 al., (1994) Nature 368:856; Taylor et al., (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al., (1990) Nature 348:552-553). Human antibodies may also be generated by in vitro activated B cells (see, e.g., U.S. Pat. Nos. 5,567,610 and 5,229,275, which are incorporated in their entirety by reference).

An “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 8(10): 1057-1062 [1995]); single chain antibody molecules; and multispecific antibodies formed from antibody fragments. The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fe[epsilon]RI. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of crosslinking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

As used herein, the term “epitope” refers to a specific arrangement of amino acids located on a protein or proteins to which an antibody binds. Epitopes often consist of a chemically active surface grouping of molecules such as amino acids or sugar side chains, and have specific three dimensional structural characteristics as well as specific charge characteristics. Epitopes can be linear or conformational, i.e., involving two or more sequences of amino acids in various regions of the antigen that may not necessarily be contiguous.

The term “diabodies” refers to small antibody fragments prepared by constructing sFv fragments (see preceding paragraph) with short linkers (about 5-10 residues) between the VH and. VL domains such that inter-chain but not intra-chain pairing of the V domains is achieved, resulting in a bivalent fragment, i.e., fragment having two antigen binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Holliger et al., Proc. Natl. Acad. Sci. USA, 90:6444-6448 (1993).

As used herein, an antibody is said to be “immunospecific”, “specific for” or to “specifically bind” an antigen if it reacts at a detectable level with the antigen, preferably with an affinity constant, Ka, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹, or greater than or equal to 10⁹ M⁻¹, or greater than or equal to 10¹⁰ M⁻¹. Affinity of an antibody for its cognate antigen is also commonly expressed as a dissociation constant Kd, and in certain embodiments, an antibody specifically binds to antigen if it binds with a Kd of less than or equal to 10⁻⁴ M, less than or equal to about 10⁻⁵ M, less than or equal to about 10⁻⁶ M, less than or equal to 10⁻⁷ M, or less than or equal to 10⁻⁸ M, or less than or equal to 5.10⁻⁹ M, or less than or equal to 10⁻⁹ M, or less than or equal to 5.10⁻¹⁰ M, or less than or equal to 10⁻¹⁰ M. Affinities of antibodies can be readily determined using conventional techniques, for example, those described by Scatchard et al., (Ann. N.Y. Acad. Sci. USA 51:660 (1949)). Binding properties of an antibody to antigens, cells or tissues thereof may generally be determined and assessed using immunodetection methods including, for example, immunofluorescence-based assays, such as immunohistochemistry (IHC) and/or fluorescence-activated cell sorting (FACS).

The term “internalize”, when use in relationship with the binding of the protein of the invention with the CD9P-1 antigen at the surface of cancer cells, refers to rapid uptake from the external milieu of the protein-ligand complex (such as, for example of the antibody-antigen complex) by receptor-mediated endocytosis, micropinocytosis, phagocytosis and other similar cellular uptake and/or trafficking pathways. In one embodiment, “Internalization” of the protein of the invention thus relates to its uptake from the external milieu by a mechanism involving plasma membrane infolding and vesicle formation. In one embodiment, said internalization involves receptor-mediated endocytosis, comprising binding of the protein of the invention to its ligand on the plasma membrane followed by trafficking of the complex protein-ligand to cytoplasmic vesicles. “Internalization” of the protein of the invention may also refers to the uptake of a CD9P-1 protein complex comprising CD9P-1 interacting proteins, such as, for example, Stabilin-1 and TRAF-2.

An “isolated nucleic acid” or “isolated nucleic sequence” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless specifically indicated otherwise. In one embodiment, as used herein, the term “peptides” refers to a linear polymer of amino acids linked together by peptide bonds, preferably having a chain length between 15 and 50 amino acids residues; a “polypeptide” refers to a linear polymer of at least 50 amino acids linked together by peptide bonds; and a protein specifically refers to a functional entity formed of one or more peptides or polypeptides, optionally glycosylated, and optionally of non-polypeptides cofactors. Therefore, in one embodiment, the term “protein” refers to a peptide as defined hereinabove. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising CDRs and being capable of binding an antigen.

An “isolated polypeptide” is one that has been identified and separated and/or recovered from a component of its natural environment. In preferred embodiments, the isolated polypeptide will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver staining. Isolated polypeptide includes the polypeptide in situ within recombinant cells since at least one component of the polypeptide's natural environment will not be present. Ordinarily, however, isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g., antibody) derived from nature (e.g., from any species). Such native sequence polynucleotides and polypeptides can be isolated from nature or can be produced by recombinant or synthetic means. A polynucleotide “variant”, as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences of the invention and evaluating one or more biological activities of the encoded polypeptide as described herein and/or using any of a number of techniques well known in the art. A polypeptide “variant”, as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating one or more biological activities of the polypeptide as described herein and/or using any of a number of techniques well known in the art. Modifications may be made in the structure of the polynucleotides and polypeptides of the present invention and still obtain a functional molecule that encodes a variant or derivative polypeptide with desirable characteristics.

When it is desired to alter the amino acid sequence of a polypeptide to create an equivalent, or even an improved, variant or portion of a polypeptide of the invention, one skilled in the art will typically change one or more of the codons of the encoding DNA sequence. For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with similar properties. It is thus contemplated that various changes may be made in the peptide sequences of the present invention, or corresponding DNA sequences that encode said peptides without appreciable loss of their biological utility or activity. In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

The term “identity” or “identical”, when used in a relationship between the sequences of two or more polypeptides or nucleic acid sequences, refers to the degree of sequence relatedness between polypeptides or nucleic acid sequences, as determined by the number of matches between strings of two or more amino acid residues or nucleotide residues. “Identity” measures the percent of identical matches between the smaller of two or more sequences with gap alignments (if any) addressed by a particular mathematical model or computer program (i.e., “algorithms”). Identity of related polypeptides or nucleic acid sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New 10 Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al., NCB/NLM/NIH Bethesda, Md. 20894; Altschul et al., supra). The well-known Smith Waterman algorithm may also be used to determine identity.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject is successfully “treated” if, after receiving a therapeutic amount of a protein according to the present invention, the subject shows observable and/or measurable reduction in the number of pathogenic cells; reduction in the percent of total cells that are pathogenic; and/or relief to some extent of one or more of the symptoms associated with the specific disease or condition; reduced morbidity and mortality, and/or improvement in quality of life issues. The above parameters for assessing successful treatment and improvement in the disease are readily measurable by routine procedures familiar to a physician.

The term “therapeutically effective amount” refers to means level or amount of agent that is aimed at, without causing significant negative or adverse side effects to the target, (1) delaying or preventing the onset of the targeted pathologic condition or disorder; (2) slowing down or stopping the progression, aggravation, or deterioration of one or more symptoms of the targeted pathologic condition or disorder; (3) bringing about ameliorations of the symptoms of the targeted pathologic condition or disorder; (4) reducing the severity or incidence of the targeted pathologic condition or disorder; or (5) curing the targeted pathologic condition or disorder. A therapeutically effective amount may be administered prior to the onset of the targeted pathologic condition or disorder for a prophylactic or preventive action. Alternatively, or additionally, the therapeutically effective amount may be administered after initiation of the targeted pathologic condition or disorder, for a therapeutic action.

The term “pharmaceutically acceptable excipient” or “pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.

The term “subject” refers to a warm-blooded animal, preferably a mammal (including humans, domestic and farm animals, and zoo, sports, or pet animals, such as dogs, cats, cattle, horses, sheep, pigs, goats, rabbits, etc. . . . ), and more preferably a human. Preferably, the subject is a patient, i.e., is awaiting the receipt of, or is receiving medical care or was/is/will be the object of a medical procedure, or is monitored for the development of a disease. In one embodiment, the subject is a male. In another embodiment, the subject is a female.

### DETAILED DESCRIPTION

One object of the invention is a protein that inhibits the CD9P-1 pathway. In one embodiment, the protein of the invention inhibits the CD9P-1stabilin-1 pathway and/or the CD9P-1TRAF-2 pathway.

As used herein, the term “inhibit the pathway” means that the protein is capable of blocking, reducing, preventing or neutralizing the signaling resulting from the binding of CD9P-1 with one of its partners, such as, for example, stabilin-1 or TRAF-2.

In one embodiment, the protein of the invention induces an internalization of CD9P-1 present at the cell surface membrane into the cytoplasm. In one embodiment, the protein of the invention further induces an internalization of Stabilin-1 present at the cell surface membrane into the cytoplasm.

Methods for determining if a protein induces an internalization of CD9P-1 and/or of Stabilin-1 are well known by the skilled artisan and include, without limitation, site-specific immunolabeling followed by flow cytometry, such as, for example, Fluorescence-Activated Cell Sorting (FACS), Laser Scanning Confocal Microscopy (LSCM) in fixed and permeabilized specimens, Fluorescence Live Cell Imaging (FLCI) or Fluorescence Recovery After Photobleaching (FRAP) in living specimens, and non-specific cell-surface chemical crosslinking (such as, for example, biotinylation) followed by immunoblotting or staining with radiolabeled antibodies, and subcellular protein fractionation through ultracentrifugation of cell membranes or sequential detergent extraction followed by immunoblotting.

In one embodiment, the protein of the invention induces the degradation of CD9P-1, and/or of Stabilin-1 and/or of TRAF-2. Methods for determining if a protein induces the degradation of CD9P-1, Stabilin-1 and/or TRAF-2 are well known by the skilled artisan and include, without limitation, western-blot, ELISA, immunoblotting, and the like.

In one embodiment, the protein of the invention destabilizes the binding of CD9P-1 and stabilin-1 and/or of CD9P-1 and TRAF-2.

In one embodiment, the protein of the invention inhibits the binding of CD9P-1 to stabi lin-1 and/or the binding of CD9P-1 to TRAF-2.

As used herein, the term “destabilizes the binding” means that the protein is capable of blocking, reducing, preventing or neutralizing the binding of CD9P-1 to stabi lin-1 and/or the binding of CD9P-1 to TRAF-2.

In one embodiment, the protein of the invention induces the dissociation of a complex CD9P-1stabilin-1 and/or of a complex CD9P-1TRAF-2.

In one embodiment, the destabilization or dissociation of the CD9P-1stabilin-1 and/or CD9P-1TRAF-2 complex is associated to or results from the internalization and/or degradation of stabilin-1 and/or TRAF-2.

Methods to detect the inhibition or destabilization of the binding of CD9P-1 to stabilin-1 and/or the binding of CD9P-1 to TRAF-2 are well-known to the skilled artisan and comprise, without limitation, immunoprecipitation, co-immunoprecipitation, immunofluorescence, crosslinking of protein complex, and chemical cross-linking followed by high-mass-resolution MALDI mass spectrometry.

In one embodiment, the protein of the invention inhibits or destabilizes the binding of CD9P-1 to stabilin-1. In one embodiment, the protein of the invention induces the degradation of CD9P-1 and/or of Stabilin-1 and/or of a complex CD9P-1/Stabilin-1. In one embodiment, the protein of the invention induces the internalization of CD9P-1 and/or of Stabilin-1 and/or of a complex CD9P-1/Stabilin-1.

Stabilin-1 (accession number NP 055951) is a transmembrane protein of 2570 amino acids expressed on subtypes of endothelial cells and activated M2 macrophages. Stabilin-1 is involved in inflammation, angiogenesis, local immunosuppression in the tumor microenvironment, tumor growth and metastasis. Stabilin-1 gene silencing or antibody treatment increases proinflammatory response including TNF-α, supports high IFN gamma production by lymphocyte and reduces tumor growth. In one embodiment, the inhibition of CD9P1/Stabilin-1 induces a reduction of cell migration and of immune tolerance to a tumor.

In another embodiment, the protein of the invention inhibits or destabilizes the binding of CD9P-1 to TRAF-2. In one embodiment, the protein of the invention induces the degradation of CD9P-1 and/or of TRAF-2 and/or of a complex CD9P-1/TRAF-2.

TRAF-2 (accession number Q12933.2) is a protein of 501 amino acids of the TNF receptor associated factor (TRAF) family, known as adapters of TNFR signaling pathway. TRAF-2 is involved in immune and inflammatory responses. TRAF-2 protein was shown as a key regulator in important aspects of immune and inflammatory responses, such as, for example, B and T lymphocyte function, NF kappa B inflammatory signaling pathway, and macrophage polarization. A loss of TRAF-2 is suggested to promote M1-like anti-tumor function of macrophages characterized by hyper expression of pro-inflammatory cytokines (such as, for example, TNF alpha and IL1 beta), to induce an anti-tumor immunity leading to tumor infiltration with IFNγ-producing CD4+ and CD8⁺ effector T cells. Moreover, TRAF-2 has anti-apoptotic signaling role through a protein complex including c-IAP proteins. TRAF-2 and TRAF-2/c-IAP depletion sensitize cancer cells to TNF-induced apoptosis. TRAF-2 was recently identified as an oncogene in epithelial cancers. Suppression of TRAF-2 in cancer cells harboring TRAF-2 overexpression inhibits proliferation, NF-κB activation, anchorage-independent growth and tumorigenesis. Without willing to be bound to any theory, the Applicant suggests that the degradation of TRAF-2 induced by the protein of the invention activates inflammatory response in macrophage and the apoptotic pathway in cancer cells.

In one embodiment, the protein of the invention is isolated.

In one embodiment, the protein of the invention binds to CD9P-1 preferably to human CD9P-1. In one embodiment, the protein of the invention binds to the extracellular domain of CD9P-1. In one embodiment, the extracellular domain or CD9P-1 corresponds to amino acids 22-832 in SEQ ID NO: 34. In another embodiment, the protein of the invention binds to an epitope comprised in a region of CD9P-1 comprising amino acids 22-724 of SEQ ID NO: 34. In another embodiment, the protein of the invention does not bind to an epitope comprised in a region of CD9P-1 comprising amino acids 724-832 of SEQ ID NO: 34.

In one embodiment, the protein of the invention binds to an epitope comprising at least one amino acid residue from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34).

In one embodiment, the epitope comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acid residues from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34).

In one embodiment, the epitope comprises at least one (e.g., 1, 2, 3, or 4) of the following residues in human CD9P-1 sequence (SEQ ID NO: 34): 211Y, 214R, 215Y and 224T.

In one embodiment, the epitope comprises the following residue in human CD9P-1 sequence (SEQ ID NO: 34): 211Y. In one embodiment, the epitope comprises the following residue in human CD9P-1 sequence (SEQ ID NO: 34): 214R. In one embodiment, the epitope comprises the following residue in human CD9P-1 sequence (SEQ ID NO: 34): 215Y. In one embodiment, the epitope comprises the following residue in human CD9P-1 sequence (SEQ ID NO: 34): 224T.

In one embodiment, the epitope comprises the following residues in human CD9P-1 sequence (SEQ ID NO: 34): 211Y and 214R. In one embodiment, the epitope comprises the following residues in human CD9P-1 sequence (SEQ ID NO: 34): 211Y and 215Y. In one embodiment, the epitope comprises the following residues in human CD9P-1 sequence (SEQ ID NO: 34): 211Y and 224T. In one embodiment, the epitope comprises the following residues in human CD9P-1 sequence (SEQ ID NO: 34): 214R and 215Y. In one embodiment, the epitope comprises the following residues in human CD9P-1 sequence (SEQ ID NO: 34): 214R and 224T. In one embodiment, the epitope comprises the following residues in human CD9P-1 sequence (SEQ ID NO: 34): 215Y and 224T.

In one embodiment, the epitope comprises the following residues in human CD9P-1 sequence (SEQ ID NO: 34): 211Y, 214R and 215Y. In one embodiment, the epitope comprises the following residues in human CD9P-1 sequence (SEQ ID NO: 34): 211Y, 214R and 224T. In one embodiment, the epitope comprises the following residues in human CD9P-1 sequence (SEQ ID NO: 34): 211Y, 215Y and 224T. In one embodiment, the epitope comprises the following residues in human CD9P-1 sequence (SEQ ID NO: 34): 214R, 215Y and 224T.

In one embodiment, the epitope comprises the following residues in human CD9P-1 sequence (SEQ ID NO: 34): 211Y, 214R, 215Y and 224T.

In one embodiment, the protein of the invention binds to an epitope comprising at least one amino acid residue from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34).

In one embodiment, the epitope comprises 1, 2, 3, 4, 5, 6, 7, 8, 9, 10, 11, 12, 13, 14, 15, 16, 17, 18, 19, 20 or 21 amino acid residues from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34).

In one embodiment, the epitope comprises at least one (e.g., 1, or 2) of the following residues in human CD9P-1 sequence (SEQ ID NO: 34): 425T and 436S.

In one embodiment, the epitope comprises the following residue in human CD9P-1 sequence (SEQ ID NO: 34): 425T. In one embodiment, the epitope comprises the following residue in human CD9P-1 sequence (SEQ ID NO: 34): 436S.

In one embodiment, the epitope comprises the following residues in human CD9P-1 sequence (SEQ ID NO: 34): 425T and 436S.

In one embodiment, the protein of the invention binds to an epitope comprising at least one amino acid residue from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, the epitope comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acid residues from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, the epitope comprises at least one (e.g., 1, 2, 3, 4 or 5) of the following residues in human CD9P-1 sequence (SEQ ID NO: 34): 472T, 474K, 478R, 497T and 501R.

In one embodiment, the epitope comprises the following residue in human CD9P-1 sequence (SEQ ID NO: 34): 472T. In one embodiment, the epitope comprises the following residue in human CD9P-1 sequence (SEQ ID NO: 34): 474K. In one embodiment, the epitope comprises the following residue in human CD9P-1 sequence (SEQ ID NO: 34): 478R. In one embodiment, the epitope comprises the following residue in human CD9P-1 sequence (SEQ ID NO: 34): 497T. In one embodiment, the epitope comprises the following residue in human CD9P-1 sequence (SEQ ID NO: 34): 501R.

In one embodiment, the epitope comprises the following residues in human CD9P-1 sequence (SEQ ID NO: 34): 472T and 474K. In one embodiment, the epitope comprises the following residues in human CD9P-1 sequence (SEQ ID NO: 34): 472T and 478R. In one embodiment, the epitope comprises the following residues in human CD9P-1 sequence (SEQ ID NO: 34): 472T and 497T. In one embodiment, the epitope comprises the following residues in human CD9P-1 sequence (SEQ ID NO: 34): 472T and 501R. In one embodiment, the epitope comprises the following residues in human CD9P-1 sequence (SEQ ID NO: 34): 474K and 478R. In one embodiment, the epitope comprises the following residues in human CD9P-1 sequence (SEQ ID NO: 34): 474K and 497T. In one embodiment, the epitope comprises the following residues in human CD9P-1 sequence (SEQ ID NO: 34): 474K and 501R. In one embodiment, the epitope comprises the following residues in human CD9P-1 sequence (SEQ ID NO: 34): 478R and 497T. In one embodiment, the epitope comprises the following residues in human CD9P-1 sequence (SEQ ID NO: 34): 478R and 501R. In one embodiment, the epitope comprises the following residues in human CD9P-1 sequence (SEQ ID NO: 34): 497T and 501R.

In one embodiment, the epitope comprises the following residues in human CD9P-1 sequence (SEQ ID NO: 34): 472T, 474K and 478R. In one embodiment, the epitope comprises the following residues in human CD9P-1 sequence (SEQ ID NO: 34): 472T, 474K and 497T. In one embodiment, the epitope comprises the following residues in human CD9P-1 sequence (SEQ ID NO: 34): 472T, 474K and 501R. In one embodiment, the epitope comprises the following residues in human CD9P-1 sequence (SEQ ID NO: 34): 472T, 478R and 497T. In one embodiment, the epitope comprises the following residues in human CD9P-1 sequence (SEQ ID NO: 34): 472T, 478R and 501R. In one embodiment, the epitope comprises the following residues in human CD9P-1 sequence (SEQ ID NO: 34): 472T, 497T and 501R. In one embodiment, the epitope comprises the following residues in human CD9P-1 sequence (SEQ ID NO: 34): 474K, 478R and 497T. In one embodiment, the epitope comprises the following residues in human CD9P-1 sequence (SEQ ID NO: 34): 474K, 478R, and 501R. In one embodiment, the epitope comprises the following residues in human CD9P-1 sequence (SEQ ID NO: 34): 474K, 497T and 501R.

In one embodiment, the epitope comprises the following residues in human CD9P-1 sequence (SEQ ID NO: 34): 474K, 478R, 497T and 501R. In one embodiment, the epitope comprises the following residues in human CD9P-1 sequence (SEQ ID NO: 34): 472T, 478R, 497T and 501R. In one embodiment, the epitope comprises the following residues in human CD9P-1 sequence (SEQ ID NO: 34): 472T, 474K, 497T and 501R. In one embodiment, the epitope comprises the following residues in human CD9P-1 sequence (SEQ ID NO: 34): 472T, 474K, 478R and 501R. In one embodiment, the epitope comprises the following residues in human CD9P-1 sequence (SEQ ID NO: 34): 472T, 474K, 478R, and 497T.

In one embodiment, the epitope comprises the following residues in human CD9P-1 sequence (SEQ ID NO: 34): 472T, 474K, 478R, 497T and 501R.

In one embodiment, the protein of the invention binds to a conformational epitope.

In one embodiment, said conformational epitope comprises at least one amino acid residue from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34).

In one embodiment, the conformational epitope comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acid residues from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34).

In one embodiment, the conformational epitope comprises at least one (e.g., 1, 2, 3, or 4) of the following residues in human CD9P-1 sequence (SEQ ID NO: 34): 211Y, 214R, 215Y and 224T.

In one embodiment, the conformational epitope comprises the following residue in human CD9P-1 sequence (SEQ ID NO: 34): 211Y. In one embodiment, the epitope comprises the following residue in human CD9P-1 sequence (SEQ ID NO: 34): 214R. In one embodiment, the epitope comprises the following residue in human CD9P-1 sequence (SEQ ID NO: 34): 215Y. In one embodiment, the epitope comprises the following residue in human CD9P-1 sequence (SEQ ID NO: 34): 224T.

In one embodiment, the conformational epitope comprises the following residues in human CD9P-1 sequence (SEQ ID NO: 34): 211Y and 214R. In one embodiment, the epitope comprises the following residues in human CD9P-1 sequence (SEQ ID NO: 34): 211Y and 215Y. In one embodiment, the epitope comprises the following residues in human CD9P-1 sequence (SEQ ID NO: 34): 211Y and 224T. In one embodiment, the epitope comprises the following residues in human CD9P-1 sequence (SEQ ID NO: 34): 214R and 215Y. In one embodiment, the epitope comprises the following residues in human CD9P-1 sequence (SEQ ID NO: 34): 214R and 224T. In one embodiment, the epitope comprises the following residues in human CD9P-1 sequence (SEQ ID NO: 34): 215Y and 224T.

In one embodiment, the conformational epitope comprises the following residues in human CD9P-1 sequence (SEQ ID NO: 34): 211Y, 214R and 215Y. In one embodiment, the epitope comprises the following residues in human CD9P-1 sequence (SEQ ID NO: 34): 211Y, 214R and 224T. In one embodiment, the epitope comprises the following residues in human CD9P-1 sequence (SEQ ID NO: 34): 211Y, 215Y and 224T. In one embodiment, the epitope comprises the following residues in human CD9P-1 sequence (SEQ ID NO: 34): 214R, 215Y and 224T.

In one embodiment, the conformational epitope comprises the following residues in human CD9P-1 sequence (SEQ ID NO: 34): 211Y, 214R, 215Y and 224T.

In one embodiment, the conformational epitope comprises at least one amino acid residue from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34).

In one embodiment, the conformational epitope comprises 1, 2, 3, 4, 5, 6, 7, 8, 9, 10, 11, 12, 13, 14, 15, 16, 17, 18, 19, 20 or 21 amino acid residues from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34).

In one embodiment, the conformational epitope comprises at least one (e.g., 1, or 2) of the following residues in human CD9P-1 sequence (SEQ ID NO: 34): 425T and 436S.

In one embodiment, the conformational epitope comprises the following residue in human CD9P-1 sequence (SEQ ID NO: 34): 425T. In one embodiment, the epitope comprises the following residue in human CD9P-1 sequence (SEQ ID NO: 34): 436S.

In one embodiment, the conformational epitope comprises the following residues in human CD9P-1 sequence (SEQ ID NO: 34): 425T and 436S.

In one embodiment, the conformational epitope comprises at least one amino acid residue from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, the conformational epitope comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acid residues from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, the conformational epitope comprises at least one (e.g., 1, 2, 3, 4 or 5) of the following residues in human CD9P-1 sequence (SEQ ID NO: 34): 472T, 474K, 478R, 497T and 501R.

In one embodiment, the conformational epitope comprises the following residue in human CD9P-1 sequence (SEQ ID NO: 34): 472T. In one embodiment, the epitope comprises the following residue in human CD9P-1 sequence (SEQ ID NO: 34): 474K. In one embodiment, the epitope comprises the following residue in human CD9P-1 sequence (SEQ ID NO: 34): 478R. In one embodiment, the epitope comprises the following residue in human CD9P-1 sequence (SEQ ID NO: 34): 497T. In one embodiment, the epitope comprises the following residue in human CD9P-1 sequence (SEQ ID NO: 34): 501R.

In one embodiment, the conformational epitope comprises the following residues in human CD9P-1 sequence (SEQ ID NO: 34): 472T and 474K. In one embodiment, the epitope comprises the following residues in human CD9P-1 sequence (SEQ ID NO: 34): 472T and 478R. In one embodiment, the epitope comprises the following residues in human CD9P-1 sequence (SEQ ID NO: 34): 472T and 497T. In one embodiment, the epitope comprises the following residues in human CD9P-1 sequence (SEQ ID NO: 34): 472T and 501R. In one embodiment, the epitope comprises the following residues in human CD9P-1 sequence (SEQ ID NO: 34): 474K and 478R. In one embodiment, the epitope comprises the following residues in human CD9P-1 sequence (SEQ ID NO: 34): 474K and 497T. In one embodiment, the epitope comprises the following residues in human CD9P-1 sequence (SEQ ID NO: 34): 474K and 501R. In one embodiment, the epitope comprises the following residues in human CD9P-1 sequence (SEQ ID NO: 34): 478R and 497T. In one embodiment, the epitope comprises the following residues in human CD9P-1 sequence (SEQ ID NO: 34): 478R and 501R. In one embodiment, the epitope comprises the following residues in human CD9P-1 sequence (SEQ ID NO: 34): 497T and 501R.

In one embodiment, the conformational epitope comprises the following residues in human CD9P-1 sequence (SEQ ID NO: 34): 472T, 474K and 478R. In one embodiment, the epitope comprises the following residues in human CD9P-1 sequence (SEQ ID NO: 34): 472T, 474K and 497T. In one embodiment, the epitope comprises the following residues in human CD9P-1 sequence (SEQ ID NO: 34): 472T, 474K and 501R. In one embodiment, the epitope comprises the following residues in human CD9P-1 sequence (SEQ ID NO: 34): 472T, 478R and 497T. In one embodiment, the epitope comprises the following residues in human CD9P-1 sequence (SEQ ID NO: 34): 472T, 478R and 501R. In one embodiment, the epitope comprises the following residues in human CD9P-1 sequence (SEQ ID NO: 34): 472T, 497T and 501R. In one embodiment, the epitope comprises the following residues in human CD9P-1 sequence (SEQ ID NO: 34): 474K, 478R and 497T. In one embodiment, the epitope comprises the following residues in human CD9P-1 sequence (SEQ ID NO: 34): 474K, 478R, and 501R. In one embodiment, the epitope comprises the following residues in human CD9P-1 sequence (SEQ ID NO: 34): 474K, 497T and 501R.

In one embodiment, the conformational epitope comprises the following residues in human CD9P-1 sequence (SEQ ID NO: 34): 474K, 478R, 497T and 501R. In one embodiment, the epitope comprises the following residues in human CD9P-1 sequence (SEQ ID NO: 34): 472T, 478R, 497T and 501R. In one embodiment, the epitope comprises the following residues in human CD9P-1 sequence (SEQ ID NO: 34): 472T, 474K, 497T and 501R. In one embodiment, the epitope comprises the following residues in human CD9P-1 sequence (SEQ ID NO: 34): 472T, 474K, 478R and 501R. In one embodiment, the epitope comprises the following residues in human CD9P-1 sequence (SEQ ID NO: 34): 472T, 474K, 478R, and 497T.

In one embodiment, the conformational epitope comprises the following residues in human CD9P-1 sequence (SEQ ID NO: 34): 472T, 474K, 478R, 497T and 501R.

In one embodiment, said conformational epitope comprises:
at least one amino acid residue from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34), and
at least one amino acid residue from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34).

In one embodiment, said conformational epitope comprises:
at least one (e.g., 1, 2, 3, or 4) of the following residues in human CD9P-1 sequence (SEQ ID NO: 34): 211Y, 214R, 215Y and 224T, and
at least one (e.g., 1, or 2) of the following residues in human CD9P-1 sequence

(SEQ ID NO: 34): 425T and 436S.

In one embodiment, said conformational epitope comprises:
the following residues in human CD9P-1 sequence (SEQ ID NO: 34): 211Y, 214R, 215Y and 224T, and
the following residues in human CD9P-1 sequence (SEQ ID NO: 34): 425T and 436S.

In one embodiment, said conformational epitope comprises:
at least one amino acid residue from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34), and
at least one amino acid residue from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, said conformational epitope comprises:
at least one (e.g., 1, 2, 3, or 4) of the following residues in human CD9P-1 sequence (SEQ ID NO: 34): 211Y, 214R, 215Y and 224T, and
at least one (e.g., 1, 2, 3, 4 or 5) of the following residues in human CD9P-1 sequence (SEQ ID NO: 34): 472T, 474K, 478R, 497T and 501R.

In one embodiment, said conformational epitope comprises:
the following residues in human CD9P-1 sequence (SEQ ID NO: 34): 211Y, 214R, 215Y and 224T, and
the following residues in human CD9P-1 sequence (SEQ ID NO: 34): 472T, 474K, 478R, 497T and 501R.

In one embodiment, said conformational epitope comprises:
at least one amino acid residue from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34), and
at least one amino acid residue from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, said conformational epitope comprises:
at least one (e.g., 1, or 2) of the following residues in human CD9P-1 sequence (SEQ ID NO: 34): 425T and 436S, and
at least one (e.g., 1, 2, 3, 4 or 5) of the following residues in human CD9P-1 sequence (SEQ ID NO: 34): 472T, 474K, 478R, 497T and 501R.

In one embodiment, said conformational epitope comprises:
the following residues in human CD9P-1 sequence (SEQ ID NO: 34): 425T and 436S, and
the following residues in human CD9P-1 sequence (SEQ ID NO: 34): 472T, 474K, 478R, 497T and 501R.

In one embodiment, said conformational epitope comprises:
at least one amino acid residue from amino acid residues 202 to 232 in human CD9P-1 (SEQ ID NO: 34), or from a sequence sharing at least 60%, 70%, 75%, 80%, 90%, 95%, 96%, 97%, 98%, 99% of identity over amino acid residues 202 to 232 of human CD9P-1 (SEQ ID NO: 34), and
at least one amino acid residue from amino acid residues 422 to 442 in human CD9P-1 (SEQ ID NO: 34), or from a sequence sharing at least 60%, 70%, 75%, 80%, 90%, 95%, 96%, 97%, 98%, 99% of identity over amino acid residues 422 to 442 of human CD9P-1 (SEQ ID NO: 34), and
at least one amino acid residue from amino acid residues 472 to 502 in human CD9P-1 (SEQ ID NO: 34), or from a sequence sharing at least 60%, 70%, 75%, 80%, 90%, 95%, 96%, 97%, 98%, 99% of identity over amino acid residues 472 to 502 of human CD9P-1 (SEQ ID NO: 34).

In one embodiment, said conformational epitope comprises:
at least one (e.g., 1, 2, 3, or 4) of the following residues in human CD9P-1 sequence (SEQ ID NO: 34): 211Y, 214R, 215Y and 224T, and
at least one (e.g., 1, or 2) of the following residues in human CD9P-1 sequence (SEQ ID NO: 34): 425T and 436S, and
at least one (e.g., 1, 2, 3, 4 or 5) of the following residues in human CD9P-1 sequence (SEQ ID NO: 34): 472T, 474K, 478R, 497T and 501R.

In one embodiment, said conformational epitope comprises:
the following residues in human CD9P-1 sequence (SEQ ID NO: 34): 211Y, 214R, 215Y and 224T, and
the following residues in human CD9P-1 sequence (SEQ ID NO: 34): 425T and 436S, and
the following residues in human CD9P-1 sequence (SEQ ID NO: 34): 472T, 474K, 478R, 497T and 501R.

In one embodiment, the protein of the invention has a KD for binding to human CD9P-1 less than or equal to about 10⁻⁵ M, preferably less than or equal to about 5.10⁻⁶ M, to about 10⁻⁶ M, to about 5.10⁻⁷ M, or less than or equal to about 10⁻⁷ M.

In one embodiment, the isolated protein has a kd for binding to human CD9P-1 of less than or equal to about 5.10⁻² sec⁻¹, preferably less than or equal to about 2.10⁻² sec⁻¹, and more preferably less than or equal to about 5.10⁻³ sec⁻¹.

In one embodiment, the isolated protein has a ka for binding to human CD9P-1 of at least about 10⁴ M⁻¹sec⁻¹, preferably at least about 5.10⁴ M⁻¹sec⁻¹.

Methods for determining the affinity (including, for example, determining the KD, ka and kd) of a protein for a ligand are well known in the art, and include, without limitation, Surface plasmon resonance (SPR, BIAcore).

In one embodiment, said protein is an antibody molecule selected from the group consisting of a whole antibody, a humanized antibody, a single chain antibody, a dimeric single chain antibody, a Fv, a Fab, a F(ab)′₂, a defucosylated antibody, a bi-specific antibody, a diabody, a triabody, a tetrabody.

In another embodiment, said protein is an antibody fragment selected from the group consisting of a unibody, a domain antibody, and a nanobody.

In another embodiment, said protein is an antibody mimetic selected from the group consisting of an affibody, an affilin, an affitin, an adnectin, an atrimer, an evasin, a DARPin, an anticalin, an avimer, a fynomer, a versabody and a duocalin.

A domain antibody is well known in the art and refers to the smallest functional binding units of antibodies, corresponding to the variable regions of either the heavy or light chains of antibodies.

A nanobody is well known in the art and refers to an antibody-derived therapeutic protein that contains the unique structural and functional properties of naturally-occurring heavy chain antibodies. These heavy chain antibodies contain a single variable domain (VHH) and two constant domains (CH2 and CH3).

A unibody is well known in the art and refers to an antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies.

An affibody is well known in the art and refers to affinity proteins based on a 58 amino acid residue protein domain, derived from one of the IgG binding domains of staphylococcal protein A.

DARPins (Designed Ankyrin Repeat Proteins) are well known in the art and refer to an antibody mimetic DRP (designed repeat protein) technology developed to exploit the binding abilities of non-antibody polypeptides.

Anticalins are well known in the art and refer to another antibody mimetic technology, wherein the binding specificity is derived from lipocalins. Anticalins may also be formatted as dual targeting protein, called Duocalins.

Avimers are well known in the art and refer to another antibody mimetic technology.

Versabodies are well known in the art and refer to another antibody mimetic technology. They are small proteins of 3-5 kDa with >15% cysteines, which form a high disulfide density scaffold, replacing the hydrophobic core the typical proteins have.

Affilins are well known in the art and refer to artificial proteins designed to selectively bind antigens. They resemble antibodies in their affinity and specificity to antigens but not in structure which makes them a type of antibody mimetic.

Adnectins, also known as monobodies, are well known in the art and refer to proteins designed to bind with high affinity and specificity to antigens. They belong to the class of molecules collectively called “antibody mimetics”.

Atrimers are well known in the art and refer to binding molecules for target protein that trimerize as a perquisite for their biological activity. They are relatively large compared to other antibody mimetic scaffolds.

Evasins are well known in the art and refer to a class of chemokine-binding proteins.

Fynomers are well known in the art and refer to proteins that belong to the class of antibody mimetic. They are attractive binding molecules due to their high thermal stability and reduced immunogenicity.

In another embodiment, said protein is a conjugate comprising the protein of the invention conjugated to an imaging agent. Said protein could be used for example for imaging applications.

In an embodiment, said protein is a monoclonal antibody.

In another embodiment, said protein is a polyclonal antibody.

In one embodiment, said protein is an isolated antibody against CD9P-1, preferably against human CD9P-1.

In one embodiment, said anti-CD9P-1 antibody binds to the 135 kDa form of CD9P-1. Without willing to be bound to any theory, the Applicant suggests that the 135 kDa form of CD9P-1 correspond to the glycosylated form of the protein.

In one embodiment, said anti-CD9P-1 antibody is capable of immunoprecipitating CD9P-1 under native conditions.

Methods for determining if an antibody is capable of immunoprecipitating CD9P-1 under native conditions are well known to the skilled artisan. A non-limiting example of such method is the following: cells are washed twice with cold PBS and lysed with 1% triton X-100 buffer for 1 hour at 4° C. Proteins are immunoprecipitated by adding the antibody of the invention; the immune complexes are harvested, for example using protein G-sepharose beads, washed with lysis buffer, resolved in SDS-PAGE and proteins were transferred to membranes (e.g., PVDF membranes) and revealed with an anti-CD9P-1 antibody.

One object of the invention is an antibody against human CD9P-1 wherein the variable region of the heavy chain comprises at least one of the followings CDRs:

| | | |
|---|---|---|
| VH-CDR1: | GYTFTSYW; | (SEQ ID NO: 1) |
| VH-CDR2: | IFPGTGTT; | (SEQ ID NO: 2) |
| and | | |
| VH-CDR3: | SRDFDV. | (SEQ ID NO: 3) |

CDR numbering and definition are according to the IMTG definition.

Another object of the invention is an antibody against human CD9P-1 wherein the variable region of the light chain comprises at least one of the followings CDRs:

| | | |
|---|---|---|
| VL-CDR1: | QSLLDIDGKTY; | (SEQ ID NO: 4) |
| VL-CDR2: | LVS; | |
| and | | |
| VL-CDR3: | WQGTHLPRT. | (SEQ ID NO: 5) |

In one embodiment of the invention, the antibody anti-CD9P-1 comprises in its heavy chain one VH-CDR1 (GYTFTSYW) (SEQ ID NO: 1), one VH-CDR2 (IFPGTGTT) (SEQ ID NO: 2) and/or one VH-CDR3 (SRDFDV) (SEQ ID NO: 3).

In another embodiment of the invention, the antibody anti-CD9P-1 comprises in its light chain one VL-CDR1 (QSLLDIDGKTY) (SEQ ID NO: 4), one VL-CDR2 (LVS) and/or one VL-CDR3 (WQGTHLPRT) (SEQ ID NO: 5).

In another embodiment of the invention, the antibody anti-CD9P-1 comprises in its heavy chain the 3 CDRs SEQ ID NO: 1, SEQ ID NO: 2 and SEQ ID NO: 3.

In another embodiment of the invention, the antibody anti-CD9P-1 comprises in its light chain the 3 CDRs SEQ ID NO: 4, LVS and SEQ ID NO: 5.

In one embodiment of the invention, the antibody anti-CD9P-1 comprises:
in its heavy chain the 3 CDRs SEQ ID NO: 1, SEQ ID NO: 2 and SEQ ID NO: 3; and
in its light chain the 3 CDRs SEQ ID NO: 4, LVS and SEQ ID NO: 5.

According to the invention, any of the CDRs 1, 2 and 3 of the heavy and light chains may be characterized as having an amino acid sequence that shares at least about 60%, 70%, 75%, 80%, 90%, 95%, 96%, 97%, 98%, 99% of identity with the particular CDR or sets of CDRs listed in the corresponding SEQ ID NO 1-5 and LVS.

In one embodiment of the invention, the antibody anti-CD9P-1 comprises the heavy chain variable region of sequence SEQ ID NO: 6.

(SEQ ID NO: 6)
QVQLQQSGAELVKPGTSVKLSCKTSGYTFTSYWIQWIKX1RPGQGLGWIG
EIFPGTGTTSYHEKFKGKATLTIDTSSSTAYLQLSNLTSEDSAVYFCSX₂
DFDVWGAGX₃X₄VTVSS;

wherein X₁ is Q or R, X₂ is R or G,X₃ is T or A and X₄ is S or T.

In one embodiment, in SEQ ID NO: 6, X₁ is R, X₂ is R,X₃ is T and X₄ is T (corresponding to the sequence SEQ ID NO:8).

In one embodiment, in SEQ ID NO: 6, X₁ is Q, X₂ is R,X₃ is A and X₄ is T (corresponding to the sequence SEQ ID NO:9).

In one embodiment, in SEQ ID NO: 6, X₁ is Q, X₂ is R,X₃ is T and X₄ is S (corresponding to the sequence SEQ ID NO:10).

In one embodiment, in SEQ ID NO: 6, X₁ is Q, X₂ is R,X₃ is T and X₄ is T (corresponding to the sequence SEQ ID NO:11).

In one embodiment, in SEQ ID NO: 6, X₁ is Q, X₂ is G,X₃ is A and X₄ is S (corresponding to the sequence SEQ ID NO:12).

In one embodiment, in SEQ ID NO: 6, X₁ is Q, X₂ is G,X₃ is A and X₄ is T (corresponding to the sequence SEQ ID NO:13).

In one embodiment, in SEQ ID NO: 6, X₁ is Q, X₂ is G,X₃ is T and X₄ is S (corresponding to the sequence SEQ ID NO:14).

In one embodiment, in SEQ ID NO: 6, X₁ is Q, X₂ is G,X₃ is T and X₄ is T (corresponding to the sequence SEQ ID NO:15).

In one embodiment, in SEQ ID NO: 6, X₁ is R, X₂ is R,X₃ is A and X₄ is S (corresponding to the sequence SEQ ID NO:16).

In one embodiment, in SEQ ID NO: 6, X₁ is R, X₂ is R,X₃ is A and X₄ is T (corresponding to the sequence SEQ ID NO:17).

In one embodiment, in SEQ ID NO: 6, X₁ is R, X₂ is R,X₃ is T and X₄ is S (corresponding to the sequence SEQ ID NO:18).

In one embodiment, in SEQ ID NO: 6, X₁ is Q, X₂ is R,X₃ is A and X₄ is S (corresponding to the sequence SEQ ID NO:19).

In one embodiment, in SEQ ID NO: 6, X₁ is R, X₂ is G,X₃ is A and.X₄ is S (corresponding to the sequence SEQ ID NO:20).

In one embodiment, in SEQ ID NO: 6, X₁ is R, X₂ is G,X₃ is A and X₄ is T (corresponding to the sequence SEQ ID NO:21).

In one embodiment, in SEQ ID NO: 6, X₁ is R, X₂ is G,X₃ is T and X₄ is S (corresponding to the sequence SEQ ID NO:22).

In one embodiment, in SEQ ID NO: 6, X₁ is R, X₂ is G,X₃ is T and X₄ is T (corresponding to the sequence SEQ ID NO:23).

Therefore, according to the invention, the heavy chain variable region of the antibody anti-CD9P-1 has a sequence that have at least about 60%, 70%, 75%, 80%, 90%, 95%, 96%, 97%, 98%, 99% of identity with SEQ ID 8-23.

In one embodiment of the invention, the antibody anti-CD9P-1 comprises the light chain variable region of sequence SEQ ID NO: 7.

(SEQ ID NO: 7)
DVVMTQTPX₅TLSVTIGQPASISCKSSQSLLDIDGKTYLNWLLQRPGQX₆
PKRLIYLVSKLDSGVPDRVTGSGSGTDFTLKIX₇RVEAEDLGVYYCWQGT
HLPRTFGGGTNLEIK;

whereinX₅ is P or L,X₆ is S or F, and X₇ is S or is absent.

In one embodiment, in SEQ ID NO: 7, Xs is L,X₆ is S, and X₇ is S (corresponding to the sequence SEQ ID NO:24).

In one embodiment, in SEQ ID NO: 7, Xs is P,X₆ is S, and X₇ is absent (corresponding to the sequence SEQ ID NO:25).

In one embodiment, in SEQ ID NO: 7, Xs is P,X₆ is F, and X₇ is S (corresponding to the sequence SEQ ID NO:26).

In one embodiment, in SEQ ID NO: 7, Xs is P,X₆ is F, and X₇ is absent (corresponding to the sequence SEQ ID NO:27).

In one embodiment, in SEQ ID NO: 7, Xs is P,X₆ is S, and X₇ is S (corresponding to the sequence SEQ ID NO:28).

In one embodiment, in SEQ ID NO: 7, X₅ is L,X₆ is S, and X₇ is absent (corresponding to the sequence SEQ ID NO:29).

In one embodiment, in SEQ ID NO: 7, X₅ is L,X₆ is F, and X₇ is S (corresponding to the sequence SEQ ID NO:30).

In one embodiment, in SEQ ID NO: 7, Xs is L,X₆ is F, and X₇ is absent (corresponding to the sequence SEQ ID NO:31).

Therefore, according to the invention, the light chain variable region of the antibody anti-CD9P-1 has a sequence that have at least about 60%, 70%, 75%, 80%, 90%, 95%, 96%, 97%, 98%, 99% of identity with SEQ ID 24-31.

In one embodiment, the anti-CD9P-1 antibody comprises a heavy chain variable region having a sequence SEQ ID NO: 6 and a light chain variable region having a sequence SEQ ID NO: 7.

In another embodiment of the invention, the anti-CD9P-1 antibody comprises a heavy chain variable region having a sequence selected from SEQ ID NO: 8-23 and a light chain variable region having a sequence selected from SEQ ID NO: 24-31.

In one embodiment, the anti-CD9P-1 antibody comprises a heavy chain variable region having a sequence SEQ ID NO: 8 and a light chain variable region having a sequence SEQ ID NO: 24.

In one embodiment, the anti-CD9P-1 antibody comprises a heavy chain variable region having a sequence SEQ ID NO: 11 and a light chain variable region having a sequence SEQ ID NO: 24.

The group of antibodies comprising a heavy chain variable region having a sequence SEQ ID NO: 6 and a light chain variable region having a sequence SEQ ID NO: 7, comprises in particular the antibodies 9bF4 and 10bB1.

Antibody 9bF4 of the invention comprises a heavy chain variable region having a sequence SEQ ID NO: 6, wherein X₁ is Q, X₂ is R,X₃ is T and X₄ is T (corresponding to the sequence SEQ ID NO:11). Clonal mutations may be observed on theX₃ position, that may be a A residue instead of a T residue. In addition, the antibody 9bF4 of the invention comprises a light chain variable region having a sequence SEQ ID NO: 7 X₅ is L,X₆ is S, and X₇ is S (corresponding to the sequence SEQ ID NO:24).

Antibody 10bB1 of the invention comprises a heavy chain variable region having a sequence SEQ ID NO: 6, wherein X₁ is Q, X₂ is R,X₃ is T and X₄ is T (corresponding to the sequence SEQ ID NO:11). Clonal mutations may be observed on the X₁ position (that may be a R residue instead of a Q residue), on the X₂ position (that may be a G residue instead of a R residue), and/or on theX₄ position (that may be a S residue instead of a T residue). In addition, the antibody 10bB1 of the invention comprises a light chain variable region having a sequence SEQ ID NO: 7 X₅ is L,X₆ is S, and X₇ is S (corresponding to the sequence SEQ ID NO:24). Clonal mutations may be observed on theX₆ position (that may be a F residue instead of a S residue), and/or on the X₇ position (that may be absent instead of being a S residue).

According to the invention, one, two, three, four or more of the amino acids of the heavy chain or light chain variable regions may be substituted by a different amino acid.

According to the invention, the heavy chain variable region encompasses sequences that have at least about 60%, 70%, 75%, 80%, 90%, 95%, 96%, 97%, 98%, 99% of identity with SEQ ID NO: 6 or 8-23.

According to the invention, the light chain variable region encompasses sequences that have at least about 60%, 70%, 75%, 80%, 90%, 95%, 96%, 97%, 98%, 99% of identity with SEQ ID NO: 7 or 24-31.

In the antibody of the invention, e.g., 9bF4 and 10bB 1, the specified variable region and CDR sequences may comprise conservative sequence modifications. Conservative sequence modifications refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis.

Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, praline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the properties set forth herein) using the assays described herein.

In one embodiment, the protein of the invention competes for binding to CD9P-1 with any one of monoclonal antibodies 9bF4 and 10bB1. In one embodiment, competition is unidirectional. In another embodiment, competition is bidirectional, which means that the protein of the invention competes for binding to CD9P-1 with any one of monoclonal antibodies 9bF4 and 10bB1, and vice versa.

In one embodiment, the protein of the invention binds to the same epitope or the same group of epitopes on CD9P -1 (preferably on the extracellular domain of CD9P-1) as any one of human monoclonal antibodies 9bF4 or 10bB1.

In the present invention, an antibody that competes (unidirectionally or bidirectionally) for binding to CD9P-1 with the 9bF4 or 10bB1 antibodies of the invention and/or binds essentially the same epitope as the 9bF4 or 10bB1 antibodies of the invention will be referred as a 9bF4 or 10bB1-like antibody, respectively.

Another object of the invention is an isolated nucleic acid sequence encoding the heavy chain variable region of sequence SEQ ID NO: 8.

In one embodiment, said nucleic acid sequence is SEQ ID NO: 32.

SEQ ID NO: 32
CAGGTCCAGCTGCAGCAGTCTGGAGCTGAACTGGTGAAGCCTGGGACTTC
AGTGAAACTGTCCTGCAAGACTTCTGGCTACACCTTCACCAGCTACTGGA
TTCAGTGGATAAAACAGAGGCCTGGACAGGGCCTTGGGTGGATTGGAGAG
ATATTTCCTGGAACTGGCACGACTTCCTACCATGAGAAATTCAAGGGCAA
GGCCACACTGACTATAGACACATCCTCCAGCACAGCCTACTTGCAGCTCA
GCAACCTGACCTCTGAAGACTCTGCTGTCTATTTCTGTTCAAGAGACTTC
GATGTCTGGGGCGCAGGCACCACTGTCACCGTCTCCTCAA.

Another object of the invention is an isolated nucleic acid sequence encoding the light chain variable region of sequence SEQ ID NO: 24.

In one embodiment, said nucleic acid sequence is SEQ ID NO: 33.

SEQ ID NO: 33
GATGTTGTGATGACCCAGACTCCACTCACTTTGTCGGTTACCATTGGGCA
ACCAGCCTCCATCTCTTGCAAGTCAAGTCAGAGCCTCTTAGATATTGATG
GAAAGACATATTTGAATTGGTTGTTACAGAGGCCAGGCCAGTCTCCAAAG
CGCCTAATCTATCTGGTGTCTAAACTGGACTCTGGAGTCCCTGACAGGGT
CACTGGCAGTGGATCAGGGACAGATTTCACACTGAAAATCAGCAGAGTGG
AGGCTGAGGATTTGGGAGTTTATTATTGTTGGCAAGGTACACATCTTCCT
CGGACGTTCGGTGGAGGCACCAACCTGGAAATCAAAC.

Another object of the invention is an expression vector comprising the nucleic acid sequences encoding the antibody anti-CD9P-1 of the invention. In one embodiment, the expression vector of the invention comprises at least one of SEQ ID NO: 32, SEQ ID NO: 33, or any sequence having a nucleic acid sequence that shares at least about 60%, 70%, 75%, 80%, 90%, 95%, 96%, 97%, 98%, 99% of identity with said SEQ ID NO: 32-33.

Another object of the invention is an isolated host cell comprising said vector. Said host cell may be used for the recombinant production of the antibodies of the invention.

Another object of the invention is a hybridoma cell line which produces the antibody of the invention.

The preferred hybridoma cell lines according to the invention were deposited by GENE SIGNAL, Genopole Industries, 4 rue Pierre Fontaine, 91000 Evry, France with the Collection Nationale de Culture de Microorganismes (CNCM), Institut Pasteur, 25 rue du Docteur Roux, 75014 Paris:

| Cell line | Deposition No. | Date of deposit |
|---|---|---|
| 9bF4 hybridoma | CNCM 1-5213 | Jul. 6, 2017 |
| 10bB1 hybridoma | CNCM 1-5214 | Jul. 6, 2017 |

In one embodiment of the invention, the antibody is a monoclonal antibody. Fragments and derivatives of antibodies of this invention (which are encompassed by the term “antibody” or “antibodies” as used in this application, unless otherwise stated or clearly contradicted by context), preferably a 9bF4-like or 10bB1-like antibody, can be produced by techniques that are known in the art. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single -chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

For instance, Fab or F(ab′)2 fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al., Cytokines 16 (3): 106-119 (2001) and Delgado et al., Br. J. Cancer 5 73 (2): 175-182 (1996), the disclosures of which are incorporated herein by reference.

Alternatively, the DNA of a hybridoma producing an antibody of the invention, preferably a 9bF4-like or 10bB1-like antibody, may be modified so as to encode a fragment of the invention. The modified DNA is then inserted into an expression vector and used to transform or transfect an appropriate cell, which then expresses the desired fragment.

In certain embodiments, the DNA of a hybridoma producing an antibody of this invention, preferably a 9bF4-like or 10bB1-like antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody of the invention.

Thus, according to another embodiment, the antibody of this invention, preferably a 9bF4-like or 10bB1-like antibody, is humanized. “Humanized” forms of antibodies according to this invention are specific chimeric immunoglobulins, immunoglobulin chains or fragments thereof (such as Fv, Fab, Fab′, F(ab′)₂, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence.

The humanized antibody optimally also will comprise at least a portion of an immunoglobulin constant region (Fc), typically that of a human immunoglobulin. For further details see Jones et al., Nature, 321, pp. 522 (1986); Reichmann et al., Nature, 332, pp. 323 (1988); Presta et al., Curr. Op. Struct. Biol., 2, pp. 593 (1992); Verhoeyen et al., Science, 239, pp. 1534; and U.S. Pat. No. 4,816,567, the entire disclosures of which are herein incorporated by reference. Methods for humanizing the antibodies of this invention are well known in the art. The choice of human variable domains, both light and heavy, to be used in making the humanized antibodies is very important to reduce antigenicity.

According to the so-called “best-fit” method, the sequence of the variable domain of an antibody of this invention is screened against the entire library of known human variable-domain sequences. The human sequence that is closest to the mouse sequence is then accepted as the human framework (FR) for the humanized antibody (Sims et al., J. Immunol. 151, pp. 2296 (1993); Chothia and Lesk, J. Mol. Biol. 196, pp. 901).

Another method uses a particular framework from the consensus sequence of all human antibodies of a particular subgroup of light or heavy chains. The same framework can be used for several different humanized antibodies (Carter et al., PNAS 89, pp. 4285 (1992); Presta et J. Immunol., 51 (1993)). It is further important that antibodies be humanized with retention of high affinity for CD9P-1 and other favorable biological properties. To achieve this goal, according to a preferred method, humanized antibodies are prepared by a process of analysis of the parental sequences and various conceptual humanized products using three-dimensional models of the parental and humanized sequences.

Three-dimensional immunoglobulin models are commonly available and are familiar to those skilled in the art. Computer programs are available which illustrate and display probable three-dimensional structures of selected candidate immunoglobulin sequences.

Inspection of these displays permits analysis of the likely role of the residues in the functioning of the candidate immunoglobulin sequence, i.e., the analysis of residues that influence the ability of the candidate immunoglobulin to bind its antigen. In this way, CDR residues can be selected and combined from the consensus and import sequences so that the desired antibody characteristic, such as increased affinity for the target antigen (s), is achieved. In general, the CDR residues are directly and most substantially involved in influencing antigen binding. Another method of making “humanized” monoclonal antibodies is to use a XenoMouse (Abgenix, Fremont, Calif.) as the mouse used for immunization. A XenoMouse is a murine host according to this invention that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et al., Nature 362 (1993) 255), or by selection of antibody repertoires using phage display methods. Such techniques are known to the skilled person and can be implemented starting from monoclonal antibodies as disclosed in the present application.

The antibodies of the present invention, preferably a 9bF4-like or 10bB1-like antibody, may also be derivatized to “chimeric” antibodies (immunoglobulins) in which a portion of the heavy/light chain(s) is identical with or homologous to corresponding sequences in the original antibody, while the remainder of the chain (s) is identical with or homologous to corresponding sequences in antibodies derived from another species or belonging to another antibody class or subclass, as well as fragments of such antibodies, so long as they exhibit the desired biological activity and binding specificity (Morrison et al., Proc. Natl. Acad. Sci. U. S. A., pp. 6851 (1984)).

Another object of the invention is a composition comprising, consisting essentially of or consisting of at least one of the protein of the invention, preferably 9bF4-like or 10bB1-like antibody.

As used herein, “consisting essentially of”, with reference to a composition, means that at least one of the protein of the invention as described here above is the only one therapeutic agent or agent with a biologic activity within said composition.

Another object of the invention is a pharmaceutical composition comprising at least one of the protein of the invention as described here above, preferably 9bF4-like or 10bB1-like antibody, and a pharmaceutically acceptable carrier.

Examples of pharmaceutically acceptable carriers include, but are not limited to, media, solvents, coatings, isotonic and absorption delaying agents, additives, stabilizers, preservatives, surfactants, substances which inhibit enzymatic degradation, alcohols, pH controlling agents, and propellants.

Examples of pharmaceutically acceptable media include, but are not limited to, water, phosphate buffered saline, normal saline or other physiologically buffered saline, or other solvent such as glycol, glycerol, and oil such as olive oil or an injectable organic ester. A pharmaceutically acceptable medium can also contain liposomes or micelles, and can contain immunostimulating complexes prepared by mixing polypeptide or peptide antigens with detergent and a glycoside.

Examples of coating materials include, but are not limited to, lecithin.

Examples of isotonic agents include, but are not limited to, sugars, sodium chloride, and the like.

Examples of agents that delay absorption include, but are not limited to, aluminum monostearate and gelatin.

Examples of additives include, but are not limited to, mannitol, dextran, sugar, glycine, lactose or polyvinylpyrrolidone or other additives such as antioxidants or inert gas, stabilizers or recombinant proteins (e.g., human serum albumin) suitable for in vivo administration.

Examples of suitable stabilizers include, but are not limited to, sucrose, gelatin, peptone, digested protein extracts such as NZ-Amine or NZ-Amine AS.

Pharmaceutically acceptable carriers that may be used in these compositions further include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylenepolyoxypropylene-block polymers, polyethylene glycol and wool fat.

Another object of the invention is a medicament comprising, consisting or consisting essentially of at least one of the proteins of the invention, preferably 9bF4-like or 10bB1-like antibody, as described hereinabove.

For use in administration to a subject, the composition will be formulated for administration to the subject. The compositions, pharmaceutical compositions and medicaments of the present invention may be administered orally, parenterally, by inhalation spray, topically, rectally, nasally, buccally, vaginally or via an implanted reservoir. The use herein includes subcutaneous, intravenous, intramuscular, intra-articular, intra-synovial, intrastemal, intrathecal, intrahepatic, intralesional and intracranial injection or infusion techniques.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added.

For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added.

Schedules and dosages for administration of the protein of the invention in the pharmaceutical compositions of the present invention can be determined in accordance with known methods for these products, for example using the manufacturers' instructions.

In the present application, the Applicant demonstrated that the protein of the invention, in particular the antibody of the invention, induces CD9P1, stabilin-1 and TRAF-2 degradation and/or internalization.

Therefore, another object of the invention is the protein of the invention for treating or for use in treating a CD9P-1-related condition, a stabilin-1 related condition and/or a TRAF-2 related condition.

The term “CD9P-1-related condition” refers to a disorder associated with an impaired expression and/or function of the CD9P-1 protein, preferably wherein inhibition of CD9P-1 can be beneficial. Accordingly, the term “stabilin-1 -related condition” refers to a disorder associated with an impaired expression and/or function of the stabilin-1 protein, preferably wherein inhibition of stabilin-1 can be beneficial, and the term “TRAF-2-related condition” refers to a disorder associated with an impaired expression and/or function of the TRAF-2 protein, preferably wherein inhibition of TRAF-2 can be beneficial.

Another object of the invention is the protein of the invention for treating or for use in treating a cancer.

Examples of cancers that may be treated by the protein, compositions and methods of the invention include, but are not limited to lung cancers, mesothelioma, breast cancers, bladder cancers, cardiac cancers, gastrointestinal cancers, genitourinary tract cancers, liver cancers, bone cancers, nervous system cancers, gynecological cancers, hematologic cancers, skin cancers, and adrenal glands cancers.

In one embodiment, said cancer is a tumor, such as, for example, a solid tumor. In another embodiment, said cancer is a blood cancer. In another embodiment, said cancer is a hematologic malignancy.

Examples of lung cancer include, but are not limited to adenocarcinoma (formerly bronchioloalveolar carcinoma), undifferentiated small cell carcinoma, undifferentiated large cell carcinoma, small cell carcinoma, large cell carcinoma, large cell neuroendocrine tumors, small cell lung cancer (SCLC), undifferentiated non-small cell lung cancer, bronchial adenoma, sarcoma, lymphoma, chondromatosis hamartoma, Pancoast tumors and carcinoid tumors.

Examples of mesothelioma include, but are not limited to pleural mesothelioma, peritoneal mesothelioma, pericardial mesothelioma, end stage mesothelioma as well as epithelioid, sarcomatous, and biphasic mesothelioma.

Examples of breast cancer include, but are not limited to ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, Paget's disease of the nipple, phyllodes tumors of the breast and recurrent & metastatic breast cancer.

Examples of bladder cancer include, but are not limited to transitional cell bladder cancer (formerly urothelial carcinoma), invasive bladder cancer, squamous cell carcinoma, adenocarcinoma, non-muscle invasive (superficial or early) bladder cancer, sarcomas, small cell cancer of the bladder and secondary bladder cancer.

Examples of cardiac cancer include, but are not limited to, sarcoma (angiosarcoma, fibrosarcoma, rhabdomyosarcoma, liposarcoma), myxoma, rhabdomyoma, fibroma, lipoma and teratoma.

Examples of gastrointestinal cancer include, but are not limited to, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, and rectal cancers.

Examples of genitourinary tract cancer include, but are not limited to, kidney (adenocarcinoma, Wihn's tumor [nephroblastoma], lymphoma, leukemia), bladder and urethra (squamous cell carcinoma, transitional cell carcinoma, adenocarcinoma), prostate (adenocarcinoma, sarcoma), and testis cancers (seminoma, teratoma, embryonal carcinoma, teratocarcinoma, choriocarcinoma, sarcoma, interstitial cell carcinoma, fibroma, fibroadenoma, adenomatoid tumors, lipoma).

Examples of liver cancer include, but are not limited to, hepatoma (hepatocellular carcinoma), cholangiocarcinoma, hepatoblastoma, angiosarcoma, hepatocellular adenoma, and hemangioma.

Examples of bone cancers include, but are not limited to, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors.

Examples of nervous system cancers include, but are not limited to, skull cancer (osteoma, hemangioma, granuloma, xanthoma, osteitis deformans), meninges cancer (meningioma, meningosarcoma, gliomatosis), and brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma).

Examples of gynecological cancers include, but are not limited to, uterus cancer (endometrial carcinoma), cervix cancer (cervical carcinoma, pre-tumor cervical dysplasia), ovaries cancer (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva cancer (squamous cell carcinoma, intraepithelial carcinoma, adenocarcinoma, fibrosarcoma, melanoma), and vagina cancer (clear cell carcinoma, squamous cell carcinoma, botryoid sarcoma [embryonal rhabdomyosarcoma], fallopian tubes cancer [carcinoma]).

Examples of hematologic cancers include, but are not limited to, blood cancer (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma].

Examples of skin cancers include, but are not limited to, malignant melanoma, basal cell carcinoma, squamous cell carcinoma, Karposi's sarcoma, moles dysplastic nevi, lipoma, angioma, dermatofibroma, and keloids.

Examples of adrenal glands cancers include, but are not limited to, neuroblastoma.

Other examples of cancers that may be treated by the protein, compositions and methods of the invention include, but are not limited to: breast, prostate, colon, ovarian, colorectal, lung, non-small cell lung, brain, testicular, stomach, pancreas, skin, small intestine, large intestine, throat, head and neck, oral, bone, liver, bladder, kidney, thyroid and blood cancer.

Other examples of cancers that may be treated by the protein, compositions and methods of the invention include, but are not limited to, lymphoma and leukemia.

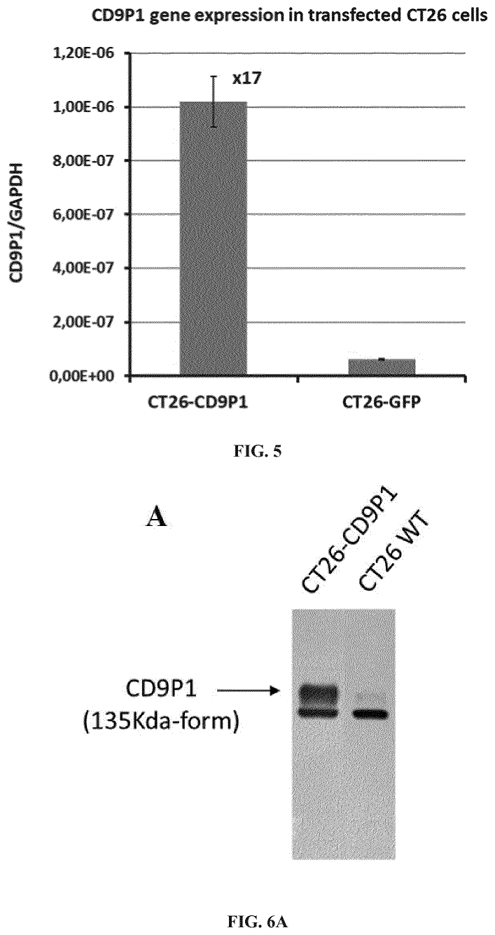

The protein, compositions and methods of the invention are also intended to prevent or decrease tumor cell metastasis. Indeed, the Applicant demonstrated that the protein of the invention induces the production of two major tumoricidal cytokines and key inducers of immune response (TNF-alpha and IFN-gamma, cf FIG. 5) in human peripheral blood mononuclear cell (PBMC) when co-cultured with human metastatic non-small cell lung carcinoma (NCI-H460) cells. Besides, the Applicant also demonstrated that the protein of the invention triggers cell apoptosis in metastatic NCI-H460 cells when cocultured with PBMC cells (cf FIG. 4).

In an embodiment, the invention concerns a protein of the invention or a composition according to the invention for use in the treatment of a cancer and/or tumor wherein cancer cells express CD9P-1.

The invention also relates to a method for treating cancer and/or tumor in a subject in need thereof wherein cancer cells and/or tumor cells express CD9P-1.

In one embodiment, the cancer cells and/or tumor cells express TRAF-2.

In one embodiment, the subject is a human.

In one embodiment, the subject has cancer. In one embodiment, the subject is diagnosed or has been diagnosed with cancer.

In one embodiment, the subject has a tumor. In one embodiment, the subject is diagnosed or has been diagnosed has having a tumor.

In one embodiment, the cancer is early or late stage cancer.

In one embodiment, the subject was not treated previously with another treatment for cancer (i.e., the method of the invention is the first line treatment).

In another embodiment, the subject previously received one, two or more other treatments for cancer (i.e., the method of the invention is a second line, a third line or more). In one embodiment, the subject previously received one or more other treatments for cancer, but was unresponsive or did not respond adequately to these treatments, which means that there is no or too low therapeutic benefit induced by these treatments.

In another embodiment, the subject is at risk of developing cancer. Examples of risk factors for developing cancer include, but are not limited to, family history of cancer, genetic predisposition, or exposure to a carcinogen.

In another embodiment of the invention, the composition comprising the protein of the invention may be used in combination with at least one other active ingredient for treating cancers and/or tumors. In one aspect of the invention, the protein or the composition comprising the protein of the invention may be used as an add-on synergistic anti-cancer agent for the treatment of cancers and/or tumors.

By “synergistic”, it is meant that the total effect of the combination of active principles is greater than the effect of each active principle taken separately. By “add-on synergistic therapy”, it is meant a combination therapy using agents with complementary mechanisms of action that improve the therapeutic effect of a monotherapy.

In a particular embodiment of the invention, the composition further comprises a cytotoxic, chemotherapeutic or anti-cancer agent.

Examples of anti-cancer agents include, but are not limited to, alkylating agents or agents with an alkylating action, such as, for example, cyclophosphamide (CTX; e.g., CYTOXAN®), chlorambucil (CHL; e.g., LEUKERANO), cisplatin (CisP; e.g., PLATINOL®), oxaliplatin (e.g., ELOXATINTM), busulfan (e.g., MYLERAN®), melphalan, carmustine (BCNU), streptozotocin, triethylenemelamine (TEM), mitomycin C, and the like; anti-metabolites, such as, for example, methotrexate (MTX), etoposide (VP16; e.g., VEPESID®), 6-mercaptopurine (6MP), 6-thiocguanine (6TG), cytarabine (Ara-C), 5-fluorouracil (5-FU), capecitabine (e.g., XELODA®), dacarbazine (DTIC), and the like; antibiotics, such as, for example, actinomycin D, doxorubicin (DXR; e.g., ADRIAMYCIN®), daunorubicin (daunomycin), bleomycin, mithramycin and the like; alkaloids, such as, for example, vinca alkaloids such as, for example, vincristine (VCR), vinblastine, and the like; and other antitumor agents, such as, for example, paclitaxel (e.g., TAXOL®) and paclitaxel derivatives, cytostatic agents, glucocorticoids such as dexamethasone (DEX; e.g., DECADRON®) and corticosteroids such as, for example, prednisone, nucleoside enzyme inhibitors such as, for example, hydroxyurea, amino acid depleting enzymes such as, for example, asparaginase, leucovorin, folinic acid, raltitrexed, and other folic acid derivatives, and similar, diverse antitumor agents. The following agents may also be used as additional agents: amifostine (e.g., ETHYOL®), dactinomycin, mechlorethamine (nitrogen mustard), streptozocin, cyclophosphamide, lornustine (CCNU), doxorubicin lipo (e.g., DOXIL®), gemcitabine (e.g., GEMZAR®), daunorubicin lipo (e.g., DAUNOXOME®), procarbazine, mitomycin, docetaxel (e.g., TAXOTERE®), aldesleukin, carboplatin, cladribine, camptothecin, 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, or chlorambucil.

The use of the cytotoxic, chemotherapeutic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In one embodiment, the composition, pharmaceutical composition or medicament of the invention comprises at least one protein of the invention, preferably a 9bF4-like or 10bB1-like antibody, and an immune checkpoint inhibitor (ICI). Various tumors are able to express molecular factors protecting them from being attacked by the immune system, and are thus capable of successfully escaping the immune system supervision control. This “tumor immune escape” is mainly due to the antagonistic blocking of receptors and binding sites targeted by immune cell ligands. Immune checkpoint inhibitors are molecules especially targeting this kind of inhibitory mechanisms developed by tumorous cells. Examples of ICIs include, but are not limited to, inhibitors of CTLA-4 (such as, for example, ipilumab and tremelimumab), inhibitors of PD-1 (such as, for example, pembrolizumab, pidilizumab, nivolumab and AMP-224) inhibitors of PD-L1 (such as, for example, atezolizumab, avelumab, durvalumab and BMS-936559), inhibitors of LAG3 (such as, for example, IMP321) and inhibitors of B7-H3 (such as, for example, MGA271).

Indeed, the Inventors demonstrate that the administration of the protein of the invention induces the production of chemoattractive molecules, thereby inducing a significant recruitment of T lymphocytes and NK cells at the site of the tumor. Without willing to be bound to any theory, the Inventors suggests that the addition of an ICI may thus allow unleashing the full immunotherapeutic potential of the method of the invention. Indeed, ICI are capable of re-establishing the immune system's capacity to attack the tumor, and therefore molecules released by immune cells recruited in the vicinity of the tumor by the protein of the invention would again efficiently bind to its target proteins at the surface of cancer cells.

Therefore, in another embodiment, the present invention relates to composition, pharmaceutical composition or medicament of the invention comprising at least one protein of the invention, preferably a 9bF4-like or 10bB1-like antibody, and an immune checkpoint inhibitor (ICI), and their use to enhance T-cell and/or NK cell function to upregulate cell-mediated immune responses and for the treatment of T cell and/or NK cell dysfunctional disorders, such as tumor immunity, for the treatment of cancer and/or tumor.

In a particular embodiment, the subject to be treated is tested or was previously tested for the presence of cancer cells expressing CD9P-1, preferably of cancer cells expressing CD9P-1 at the cell surface.

Said identification of cancer cells expressing CD9P-1 may be carried out by any method well known in the art, such as for example immunohistochemistry, PCR, or hybridization in situ, using primers, sequences or antibodies specific for CD9P-1. Examples of antibodies anti-CD9P-1 that may be used include, but are not limited to, the antibodies of the present invention, or previously described antibodies, such as, for example: SAB2700379 or HPA017074 (Sigma).

Another object of the invention is a diagnostic kit for selecting a subject in need for the treatment of the invention. In one embodiment, said diagnostic kit comprises immunoassay reagents or primers or sequences to measure the expression of CD9P-1. CD9P-1 is used as a biomarker for the companion diagnostic test. In one embodiment, said diagnostic kit comprises the CD9P-1-targeting antibody of the present invention, or previously described antibodies, such as, for example: SAB2700379 or HPA017074 (Sigma).

Another object of the invention is a method for treating cancer in a subject in need thereof, comprising:
assessing the presence of cancer cells expressing CD9P-1 in the subject;
if cancer cells expressing CD9P-1 are detected, then treating the subject by administering to the subject a protein of the invention.

Another object of the invention is a method for treating cancer in a subject in need thereof, comprising:
performing a companion diagnostic test of the subject before treatment, wherein said companion diagnostic test comprises detecting the presence of cancer cells expressing CD9P-1 using the protein of the invention, preferably the antibody of the invention;
according to a positive result of the companion diagnostic test (i.e., if CD9P-1 expressing cells are detected), treating the subject with a compound inhibiting CD9P-1.

Examples of compounds inhibiting CD9P-1 include, but are not limited to, a protein of the present invention (in particular an antibody of the present invention), and a peptide as described in WO2015/121428 (which is incorporated herein by reference), such as, for example, a peptide of sequence GNYYCSVTPWVKS (SEQ ID NO: 35) or a peptide of sequence IHSKPVFITVKMDVLNA (SEQ ID NO: 36).

Another object of the invention is the use of at least one of the protein of the invention for detecting CD9P-1 in a sample, preferably in a biological sample, in vitro or in vivo.

In one embodiment, the expression of CD9-P1 is tested in a sample comprising cancer and/or tumor cells obtained from the subject prior to the treatment.

Another object of the invention is the use of at least one of the protein of the invention for screening in vitro or in vivo molecules inhibiting CD9P-1.

Examples of assays in which the protein of the invention may be used, include, but are not limited to, ELISA, sandwich ELISA, RIA, FACS, tissue immunohistochemistry, Western-blot, and immunoprecipitation.

Another object of the invention is a method for detecting CD9P-1 in a sample, comprising contacting the sample with a protein of the invention and detecting the anti-CD9P-1 antibody bound to CD9P-1, thereby indicating the presence of CD9P-1 in the sample.

In one embodiment of the invention, the sample is a biological sample. Examples of biological samples include, but are not limited to, bodily fluids, preferably blood, more preferably blood serum, plasma, synovial fluid, bronchoalveolar lavage fluid, sputum, lymph, ascitic fluids, urine, amniotic fluid, peritoneal fluid, cerebrospinal fluid, pleural fluid, pericardial fluid, and alveolar macrophages, tissue lysates and extracts prepared from diseased tissues.

In one embodiment of the invention, the term “sample” is intended to mean a sample taken from an individual prior to any analysis. In one embodiment, the method of the present invention does not comprise a step of recovering said sample from an individual.

In one embodiment of the invention, the protein of the invention is directly labeled with a detectable label and may be detected directly. In another embodiment, the protein of the invention is unlabeled (and is referred as the first/primary antibody) and a secondary antibody or other molecule that can bind the anti-CD9P-1 antibody is labeled. As it is well known in the art, a secondary antibody is chosen to be able to specifically bind the specific species and class of the primary antibody.

The presence of anti-CD9P-1/CD9P-1 complex in the sample can be detected and measured by detecting the presence of the labeled secondary antibody. For example, after washing away unbound secondary antibody from a well comprising the primary antibody/antigen complex or from a membrane (such as a nitrocellulose or nylon membrane) comprising the complex, the bound secondary antibody can be developed and detected based on chemiluminescence of the label for example.

Labels for the anti-CD9P-1 antibody or the secondary antibody include, but are not limited to, various enzymes, prosthetic groups, fluorescent materials, luminescent materials, magnetic agents and radioactive materials. Examples of such enzymes include but are not limited to, horseradish peroxidase, alkaline phosphatase, beta-galactosidase or acetylcholinesterase; examples of prosthetic group complexes include but are not limited to, streptavidin/biotin and avidin/biotin; examples of fluorescent materials include but are not limited to, umbelliferone, fluorescein, fluorescein isothiocyanate, rhodamine, dichlorotriazinylamine fluorescein, dansyne chloride or phycoerythrin; examples of luminescent material include but are not limited to, luminal; examples of magnetic agents include gadolinium; and examples of suitable radioactive material include ¹²⁵I, ¹³¹I, ³⁵S or ³H.

Another object of the invention is the use of a protein of the invention for in vitro diagnostic assays by determining the level of CD9P-1 in subject samples. Such assays may be useful for diagnosing diseases associated with over-expression or down-expression of CD9P-1.

Another object of the invention is the use of a protein of the invention for in vitro determining the risk of a subject to develop CD9P-1 associated diseases.

Another object of the invention is the use of a protein of the invention for in vitro determining the risk of a subject to develop a CD9P-1-related condition, preferably a cancer and/or a tumor, such as, for example, cancers and/or tumors listed hereinabove.

Another object of the invention is the use of a protein of the invention for in vitro determining if a subject is likely to respond to a treatment with a protein of the invention.

The concentration or quantity of CD9P-1 present in a subject sample can be determined using a method that specifically determines the amount of CD9P-1 present. Such a method includes an ELISA method in which, for example, proteins of the invention may be conventionally immobilized on an insoluble matrix such as a polymer matrix.

Alternatively, a sandwich ELISA method can be used. Immunohistochemistry staining assays may also be used. Using a population of samples that provides statistically significant results for each stage of progression or therapy, a range of concentrations of CD9P-1 that may be considered characteristic of each stage of disease can be designated.

In one embodiment, a sample of blood or serum is taken from a subject and the concentration of CD9P-1 present in the sample is determined to evaluate the stage of the disease in the subject under study, or to characterize the response of the subject in the course of therapy. The concentration so obtained is used to identify in which range of concentrations the value falls. The range so identified correlates with a stage of disease progression or a stage of therapy identified in the various population of diagnosed subjects, thereby providing a stage in the subject under study.

One object of the invention is a sandwich ELISA method that may be used for comparing the level of bound CD9P-1 protein in a sample obtained from a subject to a threshold level to determine if the subject has a CD9P-1-related condition. As used herein, “threshold level” refers to a level of CD9P-lexpression above which a subject sample is deemed “positive” and below which the sample is classified as “negative” for the disease. A threshold expression level for a particular biomarker (e.g., CD9P-1) may be based on compilations of data from healthy subject samples (i.e., a healthy subject population). For example, the threshold expression level may be established as the mean CD9P-1 expression level plus two times the standard deviation, based on analysis of samples from healthy subjects. One of skill in the art will appreciate that a variety of statistical and mathematical methods for establishing the threshold level of expression are known in the art.

One of skill in the art will further recognize that the capture and revelation antibodies can be contacted with the sample sequentially, as described above, or simultaneously.

Furthermore, the revelation antibody can be incubated with the sample first, prior to contacting the sample with the immobilized capture antibody.

In one particular embodiment, the capture antibody is a monoclonal antibody 9bF4 or 10bB1 and the revelation antibody is another antibody binding to CD9P-1 (preferably to another epitope on CD9P-1), such as, for example, a previously described antibody, including, without limitation, SAB2700379 or HPA017074 (Sigma), more particularly such a HRP-labeled antibody. The antibodies of the invention may be used in any assay format to detect CD9P-1, including but not limited to multiplex bead-based assays.

With respect to the sandwich ELISA format described above in which two antibodies for the same biomarker (i.e., CD9P-1) are used, the capture and revelation antibodies targeting CD9P-1 should have distinct antigenic sites. By “distinct antigenic site” is intended that the antibodies are specific for different sites on the biomarker protein of interest (i.e., CD9P-1) such that binding of one antibody does not significantly interfere with binding of the other antibody to the biomarker protein. Antibodies that are not complementary are not suitable for use in the sandwich ELISA methods described above.

Another object of the invention is a kit comprising at least one protein of the invention, preferably an anti-CD9P-1 monoclonal antibody.

By “kit” is intended any manufacture (e.g., a package or a container) comprising at least one reagent, preferably an antibody, for specifically detecting the expression of CD9P-1.

In one embodiment, the kit of the invention comprises at least one antibody of the invention (e.g., 9bF4 or 10bB1) and another antibody binding to CD9P-1 (preferably to another epitope on CD9P-1), such as, for example, a previously described antibody, including, without limitation, SAB2700379 or HPA017074 (Sigma).

The kit may be promoted, distributed, or sold as a unit for performing the methods of the present invention. Furthermore, any or all of the kit reagents may be provided within containers that protect them from the external environment, such as in sealed containers.

The kits may also contain a package insert describing the kit and methods for its use.

Kits for performing the sandwich ELISA methods of the invention generally comprise a capture antibody, optionally immobilized on a solid support (e.g., a microtiter plate), and a revelation antibody coupled with a detectable substance, such as, for example HRP, a fluorescent label, a radioisotope, beta -galactosidase, and alkaline phosphatase.

In another embodiment, the detectable substance is immobilized on a solid support (e.g., a microtiter plate).

In certain embodiments, the capture antibody and the revelation antibody are anti-CD9P-1 monoclonal antibodies, particularly the capture anti-CD9P-1 monoclonal antibody designates 9bF4 or 10bB1 mAbs and the revelation antibody designates another antibody binding to CD9P-1 (preferably to another epitope on CD9P-1), such as, for example, a previously described antibody, including, without limitation, SAB2700379 or HPA017074 (Sigma). In one kit of the invention for practicing the sandwich ELISA method, the capture antibody is anti-CD9P-1 monoclonal antibody 9bF4 or 10bB1, optionally immobilized on a microtiter plate, and the revelation antibody is a HRP-labeled antibody or biotin-labeled antibody. Chemicals for detecting and quantitating the level of revelation antibody bound to the solid support (which directly correlates with the level of CD9P-1 in the sample) may be optionally included in the kit. Purified CD9P-1 may also be provided as an antigen standard.

In another embodiment, the proteins of the present invention may be used in vivo to identify tissues and organs or cells that express CD9P-1.

In one embodiment, the method comprises a step of administering a detectably labeled protein or a pharmaceutical composition comprising a detectably labeled protein to a patient in need of such a diagnostic test and a step of subjecting the patient to imaging analysis to determine the location of the protein or fragment bound-CD9P-1-expressing tissues. Imaging analysis is well known in the medical art, and includes, without limitation, X-ray analysis, magnetic resonance imaging (MRI) or computed tomography (CT).

In another embodiment of the method, a biopsy is obtained from the patient to determine whether a tissue of interest expresses CD9P-1 rather than subjecting the patient to imaging analysis.

As stated above, in an embodiment of the invention, the proteins of the invention are labeled with a detectable agent that can be imaged in a patient. For example, the protein may be labeled with a contrast agent, such as barium, which can be used for X-ray analysis, or a magnetic contrast agent, such as a gadolinium chelate, which can be used for MRI or CT. Other labeling agents include, without limitation, radioisotopes, such as (99)Tc; or other labels discussed herein. These methods may be used, e.g., to diagnose CD9P-1-mediated disorders or track the progress of treatment for such disorders.

Another object of the invention is a method for inhibiting CD9P-1 activity or the CD9P-1 pathway in a subject in need thereof, comprising administering to the subject an effective amount of the protein of the invention. In one embodiment, said method is for inhibiting the CD9P-1stabilin-1 pathway. In another embodiment, said method is for inhibiting the CD9P-1TRAF-2 pathway. In one embodiment, the protein of the invention is an antibody (preferably a monoclonal antibody) directed to CD9P-1 as described hereinabove.

In one embodiment, the CD9P-1 antibody of the invention modulates, e.g., blocks, inhibits, reduces, antagonizes, neutralizes or otherwise interferes with the interaction between CD9P-1 and Stabilin-1 and/or TRAF-2.

In one embodiment, the CD9P-1 antibody of the invention is capable of modulating, e.g., blocking, inhibiting, reducing, antagonizing, neutralizing or otherwise interfering with CD9P-1 expression, activity and/or signaling, or of modulating proteins interacting with CD9P-1 (preferably of proteins binding to CD9P-1), such as, for example Stabilin-1 and TRAF-2.

Another object of the invention is a method for inducing internalization and/or degradation of CD9P-1, Stabilin-1 and/or TRAF-2 in a subject in need thereof, comprising administering to the subject an effective amount of the protein of the invention.

In one embodiment, the CD9P-1 antibody of the invention binds to CD9P-1 on the cell surface and causes internalization and/or degradation of CD9P-1/Stabilin-1 complex and/or CD9P-1/TRAF-2 complex.

Another object of the invention is a method for inducing an immune response and/or an inflammatory response in a subject in need thereof (preferably wherein the subject has cancer), comprising administering to the subject an effective amount of the protein of the invention. In one embodiment, the protein of the invention is an antibody (preferably a monoclonal antibody) directed to CD9P-1 as described hereinabove. In one embodiment, the method is for activating the innate immune system. In another embodiment, the method is for activating the adaptive immune system. In another embodiment, the method is for activating both the innate and the adaptive immune systems.

In one embodiment, the method of the invention is for inhibiting the polarization of immunosuppressive M2-type macrophages and/or induce pro-inflammatory M1-type macrophages.

“M1 -type macrophage” refers to immune effector cells with an acute inflammatory phenotype. These are highly aggressive against cancer cells and produce large amounts of cytokines. “M2-type macrophage” refers to anti-inflammatory macrophages having various different functions, including regulation of immunity, maintenance of immune tolerance and tissue repair. Commonly accepted marker profile for M1 -macrophages include, but are not limited to, TNFα and CD80, whereas M2-macrophages are characterized as expressing in particular CD163.

In one embodiment, the protein of the invention stimulates the pro-inflammatory function of M1-type macrophages and/or inhibits the M2-type macrophage profile.

In one embodiment, the method of the invention is for inducing TNF-alpha production in human monocytes and M2 macrophages.

In one embodiment, the method of the invention is a method for inducing M2 macrophages repolarization in M1 macrophages.

In one embodiment, the protein of the invention stimulates lymphocyte proliferation and/or stimulates T helper cells (Th1), cytotoxic T lymphocyte (CTL) and/or Natural killer (NK) cell responses, including for example, the production and release of Granzyme B and IFN gamma, and the induction of co-stimulatory proteins as B7.1 (CD80) on antigen-presenting cells (APC).

In one embodiment, the protein of the invention (preferably the CD9P-1 antibody of the invention) induces the production of Granzyme B by immune cells including, without limitation, cytotoxic T lymphocyte (CTL), Natural killer (NK), macrophages and activated microglia, and/or stimulates the Granzyme B-mediated death of cancer cells.

In one embodiment, the protein of the invention induces an increased production of CD80 by APC, thereby inducing the activation of T cells.

In one embodiment, the protein of the invention (preferably the antibody of the invention) triggers the production of chemoattractive molecules influencing the biodistribution of immune cells at the site of the tumor. In one embodiment, chemoattractive molecules comprise, but are not limited to, classical chemoattractants and chemokines.

Examples of classical chemoattractants comprise, but are not limited to, GM-CSF (Granulocyte Macrophage Colony Stimulating Factor), MCP1 (Monocyte Chemoattractant Protein-1), RANTES (Regulated on Activation Normal T Expressed and Secreted), CXCL12/SDF (Stromal cell-Derived Factor 1), MIF (Macrophage migration Inhibitory Factor) and the like.

In one embodiment, chemokines are specific cytokines, i.e., signaling molecules released by immune cells, that functions by attracting immune cells to sites of inflammation (i.e., inducing chemotaxis). Chemokines: are 70-100 aa secreted proteins (8-14 kDa) that display a 20-70% similarity in their primary sequences, and include four conserved cysteine residues in their sequences. They form four distinct families termed CC, CXC, XC and CX, where C represents the cysteine residue and X denotes any intervening amino acids between the C residues. In one embodiment, chemokines of the CC family comprise, but are not limited to, CCL 1 to CCL28. In one embodiment, chemokines of the CXC family comprise, but are not limited to, CXCL1 to CXCL17. CXCL-8 is also called interleukine 8 (IL-8). In one embodiment, chemokines of the XC family comprise, but are not limited to, XCL1 to XCL3. In one embodiment, chemokines of the CX family comprise, but are not limited to, CX3CL1.

In one embodiment, the method of the invention is for inducing TNF-alpha and IFN-gamma production in human lymphocytes.

Another object of the invention is a method for inducing apoptosis of cancer cells (such as, for example, of CD9P-1-expressing cancer cells) in a subject in need thereof, comprising administering to the subject an effective amount of the protein of the invention. In one embodiment, apoptosis of cancer cells results from the internalization and degradation of TRAF-2 induced by the protein of the invention.

Another object of the invention is a method for inhibiting the proliferation of cancer cells (such as, for example, of CD9P-1-expressing cancer cells) in a subject in need thereof, comprising administering to the subject an effective amount of the protein of the invention.

Another object of the invention is a method for inhibiting tumor growth (such as, for example, of CD9P-1-expressing cancer cells) in a subject in need thereof, comprising administering to the subject an effective amount of the protein of the invention.

Another object of the invention is a method for treating a CD9P-1 and/or stabilin-1 and/or TRAF-2 related condition in a subject in need thereof, comprising administering to the subject an effective amount of the protein of the invention. In one embodiment, said CD9P-1 and/or stabilin-1 and/or TRAF-2 related condition is cancer and/or tumor.

Another object of the invention is a method for treating a cancer in a subject in need thereof, comprising administering to the subject an effective amount of the protein of the invention.

In one embodiment, the protein of the invention (preferably the CD9P-1 antibody of the invention) allows to treat cancer through either an indirect pathway involving the immune system (i.e., inducing an inflammatory and/or immune response) and/or a pathway involving CD9P-1-expressing cancer cell apoptosis and resulting from the internalization and degradation of TRAF-2.

In one embodiment, the protein of the invention induces Antibody-dependent cell-mediated cytotoxicity (ADCC), thereby allowing treating cancer. Therefore, in one embodiment, the protein of the invention (preferably the CD9P-1 antibody of the invention) allows to treat cancer through ADCC involving CD9P-1-expressing cancer cells.

### BRIEF DESCRIPTION OF THE D WINGS

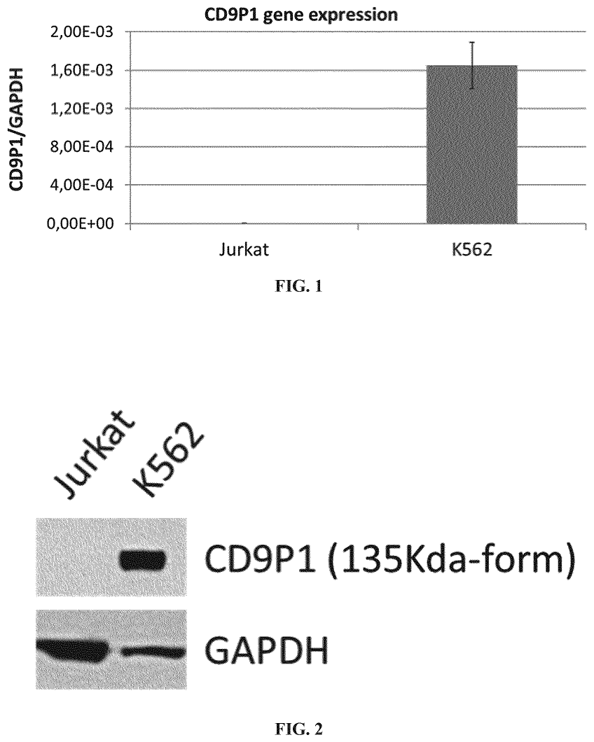

FIG. 1 is a histogram depicting CD9P-1 mRNA expression in Jurkat and K562 cell lysates as quantified by QPCR. Jurkat cells are immortalized T-cell acute lymphoblastic leukemia cell lines (T-ALL), and K562 cells are immortalized myelogenous leukemia line cells (CML). K562 cells express CD9P-1 while Jurkat cells do not. The housekeeping gene GAPDH was used as an internal control for QPCR.

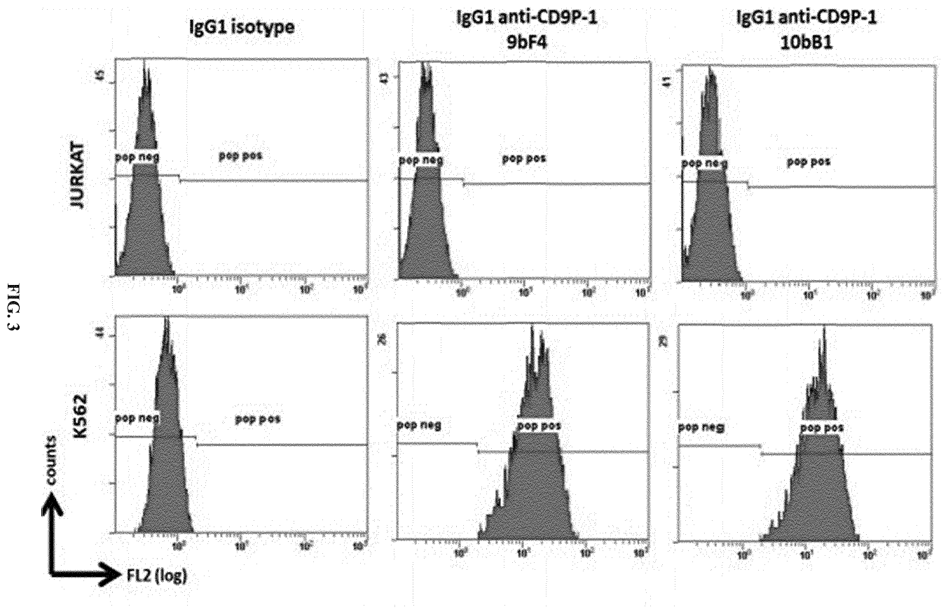

FIG. 2 is a photograph of Western Blots showing the expression of the CD9P-1 protein in Jurkat and K562 cell lysates using an anti-CD9P-1 monoclonal antibody. K562 cells express CD9P-1 while Jurkat cells do not. The housekeeping gene GAPDH was used as an internal control for western blotting.

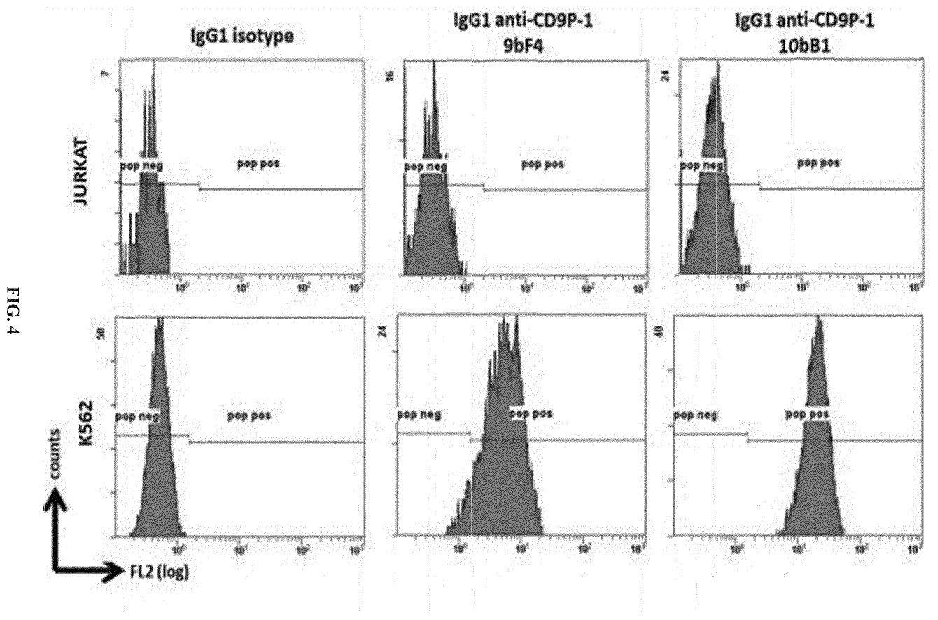

FIG. 3 are histograms showing the screening of hybridomas supernatants on K562 and Jurkat cell lines by flow cytometry. In brief, surface CD9P-1 protein was detected with supernatants of hybridomas and a PE-conjugated goat-anti-mouse antibody. Cell-surface of K562 cells was stained with 9bF4 and 10bB1 hybridoma supernatant while Jurkat cells was not.

FIG. 4 are histograms showing the quantification of CD9P-1 surface expression on K562 and Jurkat cell lines as measured by flow cytometry. Shortly, surface CD9P-1 protein was detected with the monoclonal antibody (isotype or anti-CD9P-1 antibodies) and a PE-conjugated goat anti-mouse antibody. Cell-surface of K562 cells was stained with 9bF4 and 10bB1 purified antibody while Jurkat cells did not.

FIG. 5 is a histogram depicting CD9P-1 mRNA expression in CD9P-1-transfected CT26 cell lysates as quantified by QPCR. CT26 cells are immortalized colon carcinoma line cells. CD9P-1-transfected CT26 cells express CD9P-1 while GFP-transfected CT26 cells do not. GFP-transfected CT26 and non-transfected CT26 cells were used as control for transfection. The housekeeping gene GAPDH was used as an internal control for QPCR.

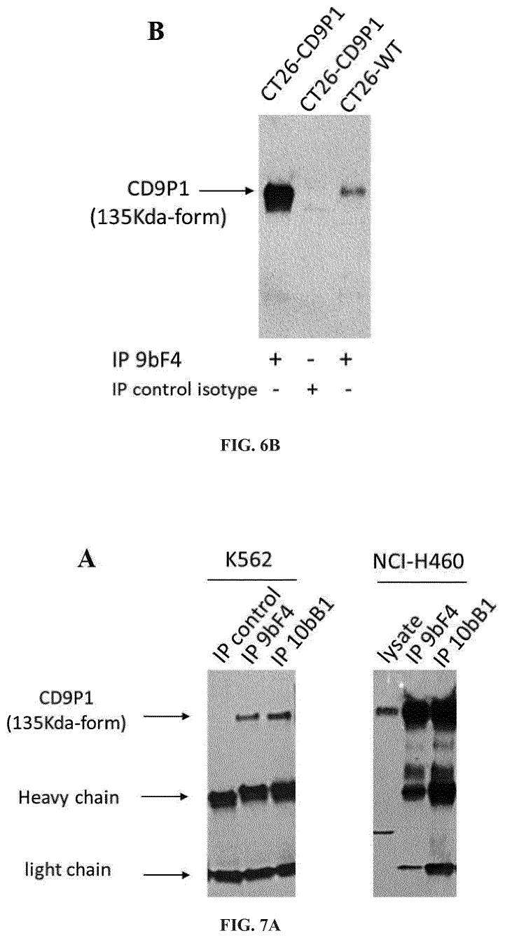

FIG. 6 is a set of photographs of Western Blots. (A) photographs of Western Blots showing the expression of the CD9P-1 protein in CD9P-1-transfected CT26 cell lysates using an anti-CD9P-1 monoclonal antibody. CD9P-1-transfected CT26 cells express CD9P-1 while non-transfected CT26 cells do not. (B) photographs of Western Blots showing the expression of the CD9P-1 protein in CD9P-1-transfected (or non-transfected) CT26 cell lysates immunoprecipitated (IP) with the 9bF4 mAb of the invention. An IgG isotype antibody was used as IP negative control. The 135 kDa form of CD9P-1 immunoprecipitated by 9bF4 from CD9P1-overexpressing CT26 cells was detected by immunoblot with a CD9P-1-targeting mAb.

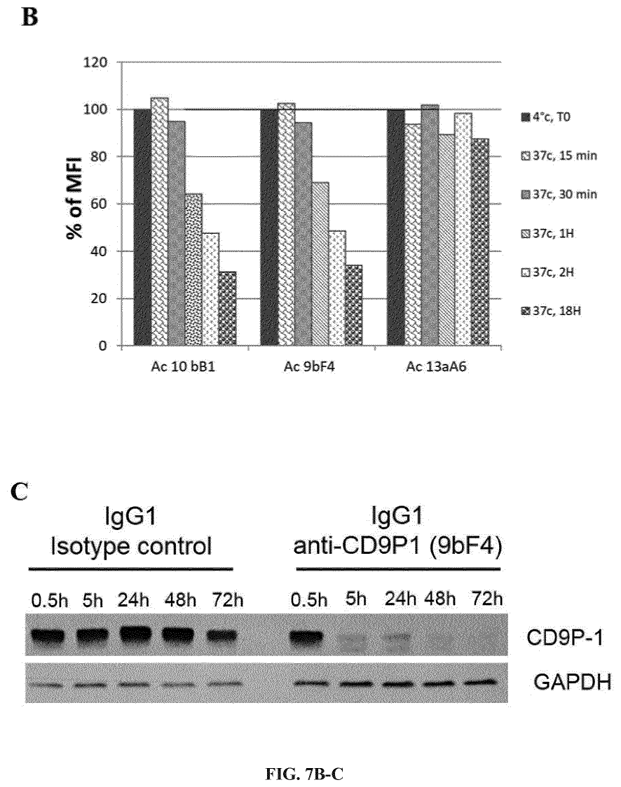

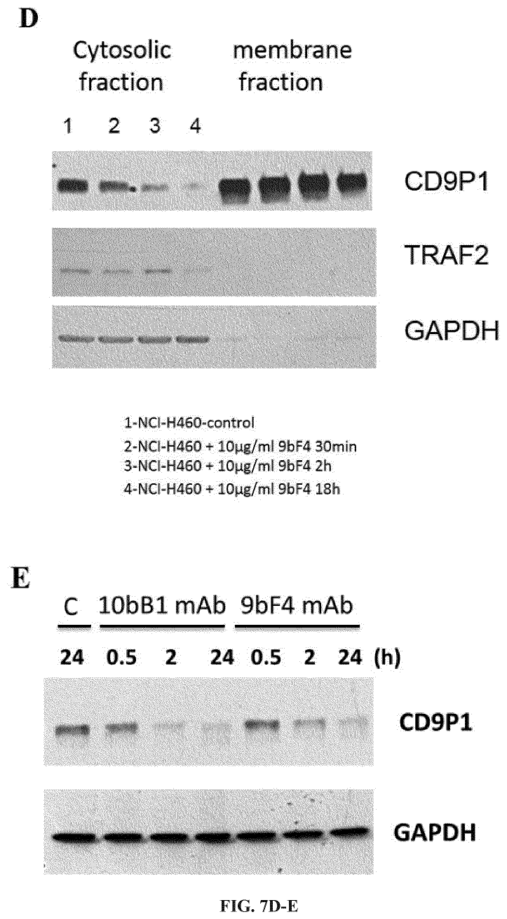

FIG. 7 is a set of histograms and photographs of Western Blots. (A) photographs of Western Blots showing the expression of the CD9P-1 protein in K562 and NCI-H460 cell lysates, respectively immunoprecipitated with: 1) the 9bF4 and 10bB1 hybridoma supernatants, and 2) the 9bF4 and 10bB1 purified mAbs. An IgG isotype antibody was used as IP negative control. Samples were separated by SDS-PAGE and immunoblotted with a CD9P-1-targeting mAb. The 135k Da form of CD9P-1 was immunoprecipitated by 9bF4 and 10bB1 mAbs in the two CD9P1-expressing human cancer cell lines. The supernatant of a non-producing hybridoma was used as a western blot control; (B) histograms depicting the analysis of CD9P-1-targeting mAbs internalization in K562 leukemia cells by flow cytometry. In brief, K562 cells were treated with various CD9P-1-targeting antibodies (9bF4, 10bB1 and 13aA6) at 4° C., washed and then incubated at 37° C. for internalization. After different time periods (0, 15, 30 min and 1, 2, 18 h), cells were collected and stained with PE-labeled secondary antibody at 4° C. and the corresponding cell surface signals were analyzed with a cytometer. The results show that K562 cells internalized 51% (9bF4) and 52% (10bB1) of cell surface-bound antibodies within 2 h and, respectively, 66% and 69% at 18 h while only 13% of 13aA6 mAb was internalized at 18 h; (C) photographs of Western Blots showing that 9bF4 mAb drastically decreased CD9P-1 expression in K562 cells after 5,24, 48 and 72 hours of incubation; (D) photographs of Western Blots showing that 9bF4 drastically decreased CD9P1 expression in membrane and cytosolic fractions of NCI-H460 cells, thus indicating that 9bF4 induces a rapid internalization and degradation of CD9P1 in these cells; (E) photographs of Western Blots showing that 9bF4 and 10bB1 mAb drastically decreased CD9P-1 expression in K562 cells after 2 hours of incubation (C: control).

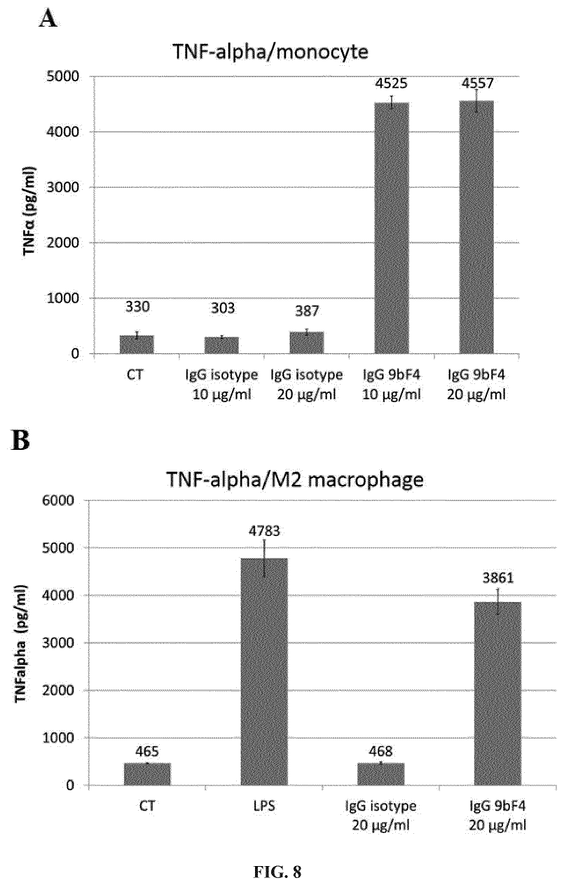

FIG. 8 are histograms showing human monocytes (A) and M2 polarized macrophages (B) increased secretion of TNF-alpha in response to antibody against human CD9P-1. The histograms display the TNF-alpha concentration in the supernatant (in pg/ml) as a function of the antibody tested. TNF-alpha concentration was measured by Sandwich ELISA. Results obtained for a CD9P-1-targeting antibody (Mouse IgG 9bF4, 10-20 μg per ml) and an IgG isotype control are compared. LPS was used as a trigger of M2 to M1 macrophage polarization switch.

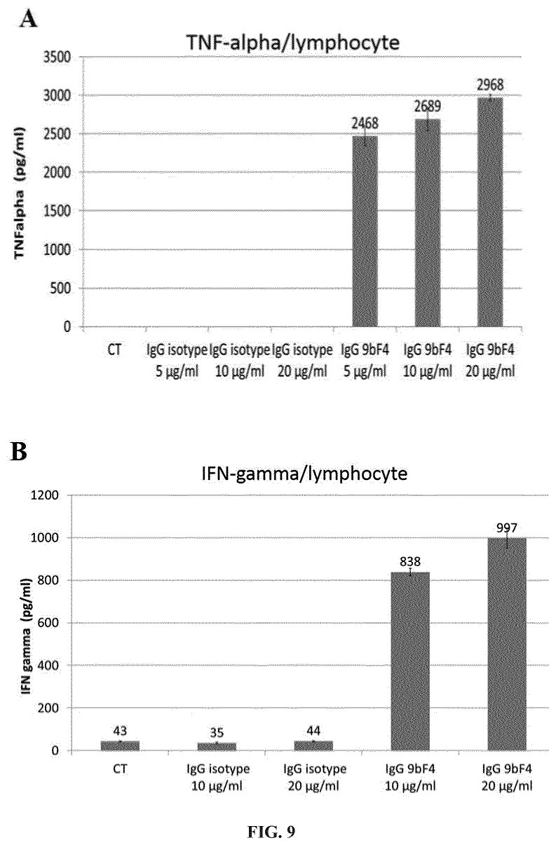

FIG. 9 are histograms showing human lymphocytes increased secretion of TNF-alpha (A) and IFN-gamma (B) in response to antibody against human CD9P-1. The histograms display TNF-alpha and IFN-gamma concentrations in the supernatant (in pg/ml) as a function of the antibody tested. TNF-alpha and IFN-gamma concentrations were measured by Sandwich ELISA. Results obtained for a CD9P-1-targeting 1 antibody (Mouse IgG 9bF4, 5-20 μg per ml) and an IgG isotype control are compared.

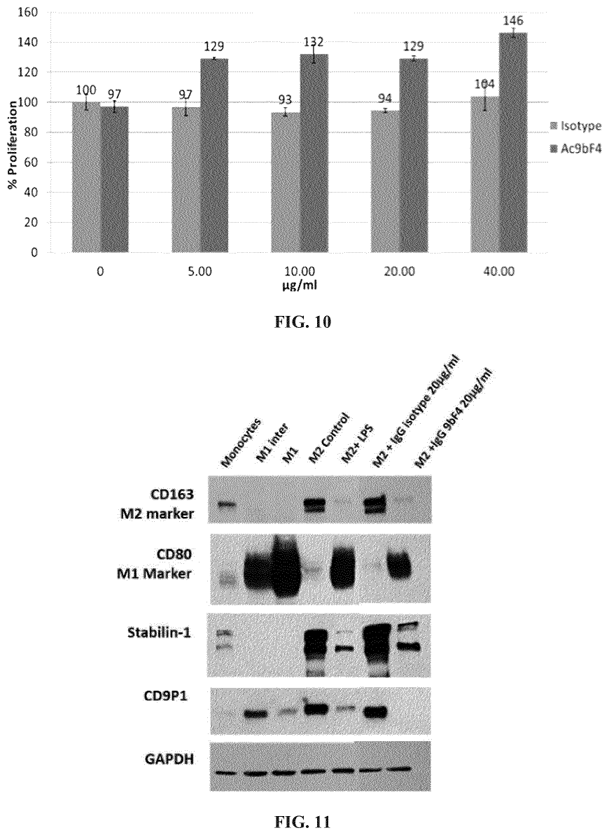

FIG. 10 is a histogram showing the increased percentage of human lymphocyte proliferation as a function of the concentration of antibody against human CD9P-1 used for incubation as quantified by MTT assay. Results obtained for a CD9P-1-targeting antibody (Ac9bF4, 0-40 μg per ml) and an IgG isotype control are compared.

FIG. 11 is a photograph of Western Blots showing the expression of M1/M2 macrophage (CD80/CD163) and M2-polarized macrophage (Stabilin-1) molecular markers over M1- or M2-differentiated PBMC lysates. M2-differentiated PBMC were pre-incubated for 48 h with a CD9P-1-targeting antibody (Mouse IgG 9bF4, 20 μg per ml) or its IgG isotype control. CD9P-1 antibody induces M2 macrophages repolarization in M1 macrophages through stabilin-1 pathway. GAPDH was used as a loading control for western blotting and LPS was used as a trigger of M2 to M1 macrophage polarity shift.

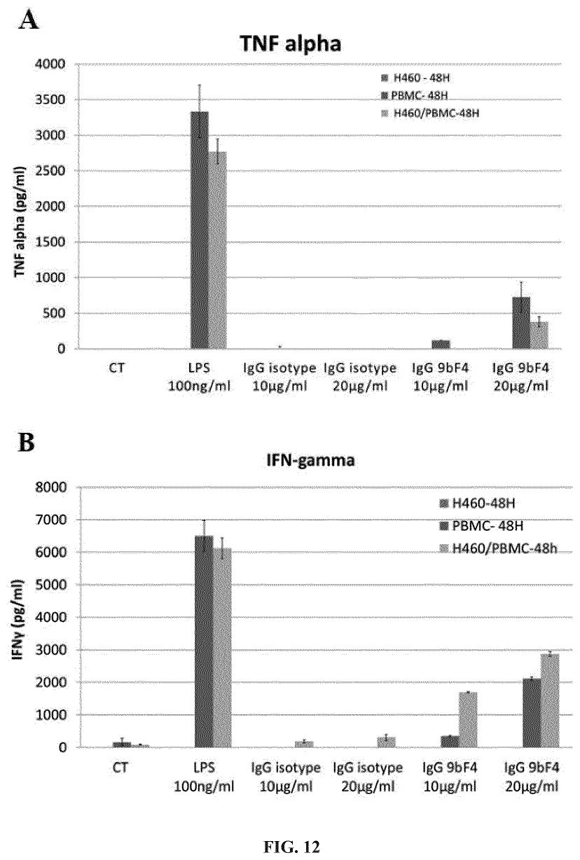

FIG. 12 are histograms showing human PBMC increased secretion of TNF-alpha (A) and IFN-gamma (B) in response to antibody against human CD9P-1. PBMC were co-cultured with human cancer cells (NCI-H460) for 48 h. The histograms display TNF-alpha and IFN-gamma concentrations in the supernatant (in pg/ml) as a function of the antibody tested. TNF-alpha and IFN-gamma concentrations were measured by Sandwich ELISA. Results obtained for a CD9P-1-targeting antibody (Mouse IgG 9bF4, 10-20 μg per ml) and an IgG isotype control are compared. LPS was used as a trigger of M2 to M1 macrophage polarization switch.

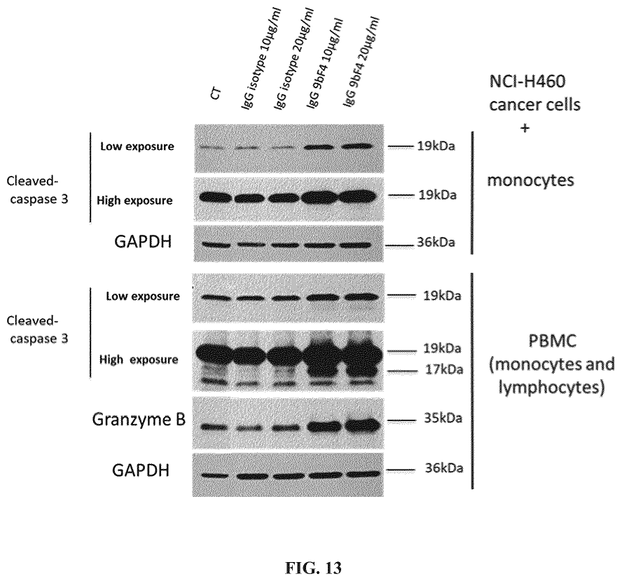

FIG. 13 is a photograph of Western Blots showing the expression of cleaved-caspase 3 (17-19 kDa) and granzyme B over monocytes or PBMC lysates. Monocytes and PBMC were co-cultured with human cancer cells (NCI-H460) for 48 h and pre-incubated with a CD9P-1-targeting antibody (Mouse IgG 9bF4, 10-20 μg per ml) or its IgG isotype control. CD9P-1 antibody induces CD9P-1-expressing cancer cells apoptosis, most probably through monocyte and lymphocyte activation as shown by the increased expression of Granzyme B, a major inducer of apoptosis released by immune cells. Samples were normalized to GAPDH.

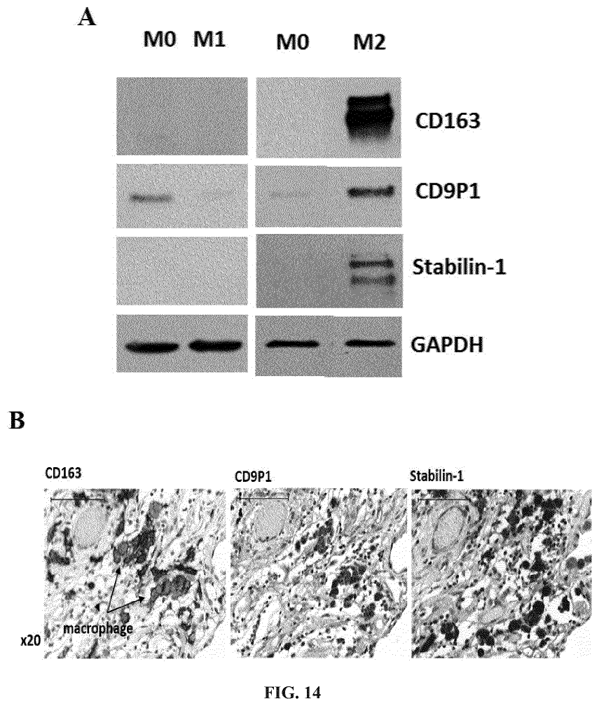

FIG. 14 is a set of photographs. (A) photograph of Western Blots showing the expression of Stabilin-1 and CD9P-1 over human M0 or M1/M2 macrophage cell lysates. CD9P-1 and Stabilin-1 are over-expressed in M2 macrophages compared to M0/M1 macrophages. GAPDH was used as a loading control for western blotting and CD163 was used as a M2-polarized marker. (B) photograph showing the co-expression of CD9P-1 and Stabilin-1 in human lung adenocarcinoma (ypT 1 aN2) after chemotherapy with cisplatin and permetrexed (Alimta^{Tm}). CD9P-1 and Stabilin-1 expression level and localization are evidenced by immunohistochemical staining, with a M2 macrophage marker (CD163) and scanned with the Lamina scanner (Perkin Elmer) SCAN (zoom x20).

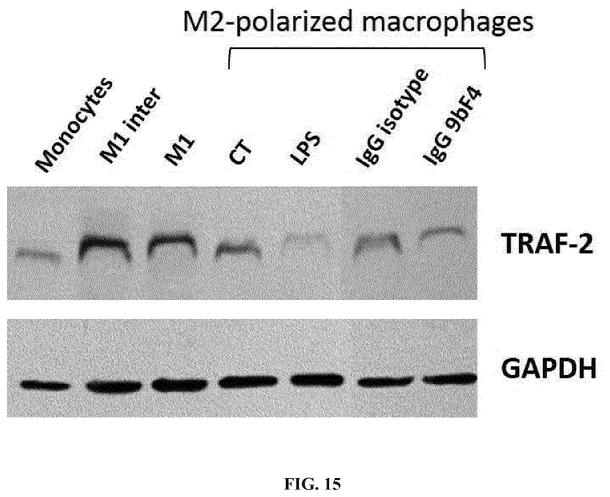

FIG. 15 is a photograph of Western Blots depicting the expression of TNF receptor-associated factor 2 (TRAF-2) in monocytes and M1- or M2-differentiated PBMC lysates. M2-differentiated PBMC were incubated for 48 h with an anti-CD9P-1 antibody (Mouse IgG 9bF4, 20 μg per ml) or its IgG isotype control. 9bF4 mAb decreased TRAF-2 expression in M2-polarized macrophages. GAPDH was used as loading control for western blot and LPS was used as an inducer of M1 polarization.

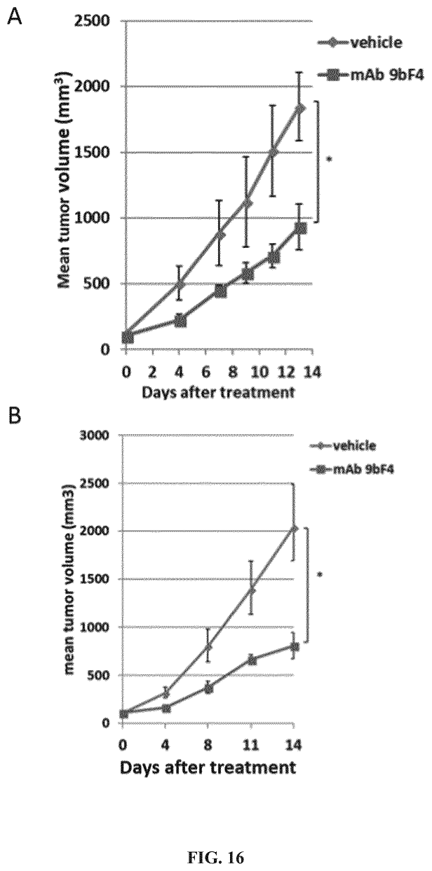

FIG. 16 is a combination of graphs showing the in vivo effect of a mouse anti-hCD9P1 antibody (9bF4 mAb) on tumor growth in nude mice engrafted with human cancer cells: (A) K562 cells and (B) NCI-H460 cells.

### EXAMPLES

The present invention is further illustrated by the following examples.

Materials and Methods

Production and Characterization of Monoclonal Antibodies Recognizing CD9P-1

To produce mAb directed to CD9P-1, we immunized mice (Balbc, Harlan, Gannat, France) four times with a purified recombinant protein corresponding to CD9P-1 extracellular domain (amino acid 22-832, accession number NP_065173.2) produced in CHO expression system (Evitria, Zurich, Switzerland). Monoclonal hybridomas were isolated by a semi-solid cloning method using ClonaCell™-HY kit (STEMCELL Technologies, Grenoble,France). Hybridomas producing anti-CD9P-1 mAbs were selected: 1) by direct ELISA of supernatant with Strep-CD9P1-ECD (wherein ECD stands for extracellular domain) used as an antigen coated onto plastic wells; and 2) by flux cytometry on the basis of the reactivity of the supernant toward CD9P-1-expressing cells (K562) and no reactivity toward CD9P-1-non-expressing cells (Jurkat) (FIGS. 1-3). K562, human Chronic Myelogenic Leukemic cell line (ATCC® CCL-243™) was maintained in RPMI containing 10% FCS at 37° C. and 5% CO₂ humidified atmosphere. After purification by affinity with Hitrap Protein G HP (GE healthcare, Velizy-Villacoublay, France) from hybridoma supernatants, anti-CD9P-1 mAbs were identified for their ability to bind CD9P-1 on the surface of living cells by cytometry (FIG. 4) and immunoprecipitate the 135 kDa-form of CD9P-1 reported previously by Charrin et al., (2001). The cell line CT26. (murine colon carcinoma, ATCC® CRL-2638TH), weakly expressing CD9P-1, were transfected with pcDNA3.1-CD9P-1 from human cDNA sequence, accession number NM_020440.3 (Genecust, Dudelange, Luxembourg) using Lipofectamine 3000 according to the manufacturer's instructions (Life Technologies, Saint-Aubin, France). A cell line stably overexpressing CD9P-1 was established in semi-solid medium with 500 μg/ml of G418 (Sigma-Aldrich, Saint Quentin Fallavier, France). Following characterization by QPCR and western blot, the selected positive clonal cells were cultured under the G418 selection-medium for approximately 3 weeks. Then CT26-CD9P-1 cells were maintained in the culture medium containing 250 μg/ml G418. Cell lines were cultured in RPMI supplemented with 10% FCS, 2 mM glutamine, 10 mM Hepes, 1 mM sodium and G418. Cells were maintained in a 37° C. humidified incubator in the presence of 5% CO₂. 9bF4 mAb specificity was controlled in immunoprecipitation experiments. CT26-CD9P-1 and CT26-WT cells were washed two times with cold PBS and lysed directly in the tissue culture flask (2 ml for a 75-cm2 flask) in 1% Triton X100 CST cell lysis buffer (Ozyme, Montigny-le-Bretonneux, France), containing 1 mM phenyl-methylsulfonyl fluoride, 1μg/ml leupeptin, 1 μl/ml pepstatin A, and 1 μg/ml Aprotinin. After a 30-min incubation at 4° C., the insoluble material was removed by centrifugation at 14,000 g for 10 min at 4° C., and protein concentrations were determined by Bradford assay. The cell lysates were precleared 1 hour by addition of 20 μl Agarose control beads and immunoprecipitated with 6μg of 9bF4 mAb coupled with agarose bead (10 μl) using Pierce Crosslink Immunoprecipitation Kit according to the manufacturer's instructions (Thermo Scientific, Courtaboeuf, France). The 135 kDa-form of CD9P-1 was then detected by western blot with the anti-CD9P-1 mAb (229t mAb) reported by Guilmain et al., (2011) (FIGS. 5-6A). 9bF4 and 10bB1 specificity by immunoprecipitation were also tested in the CD9P-1-expressing human cancer cells K562 and NCI-H460. 9bF4 and 10bB1 hybridoma supernatants were used to immunoprecipitate CD9P-1 from K562 cell lysates. 500 ᵢll of supernatant was incubated with lmg of protein and 10 μl of agarose beads. 9bF4 and 10bB1 purified mAbs were used to immunoprecipitate CD9P-1 from NCI-H460 cell lysates (FIGS. 6B-7).

Human PBMC Isolation and Monocyte/Lymphocyte Separation

Human peripheral blood was collected from healthy adult volunteers (EFS, France). Peripheral blood mononuclear cells (PBMCs) were isolated from heparinized blood by density gradient centrifugation using Lymphoprep (1114545, Stemcell Technologies, Grenoble, France). Cells were directly used for some experiments or for subsequent isolation of monocytes and lymphocytes. The monocytes (potentially including dendritic cells) were separated from the lymphocytes (potentially including NK cells) by adherence method. PBMCs were cultured at 37° C. in a humidified incubator with 5% CO2 in plastic plates in RPMI-1640 containing 100 U/ml penicillin, 100 μg/ml streptomycin, and 10% of heat inactivated fetal bovine serum during 18H. After incubation, adherent monocytes were isolated from detached lymphocytes cells. Monocytes and lymphocytes were maintained in the same medium and used separately in different experiments. In some experiments, monocytes were isolated from PBMCs by positive selection of CD14+ cells using a MACS system (Miltenyi Biotec, Paris, France), according to the manufacturer's protocol to confirm the results.

Macrophage Polarization

After monocyte isolation by adherence from 10×10⁶ PBMCs in six-well plates, non-adherent cells were removed and the medium was changed after 2 washes. M2 macrophage phenotype was obtained by adding M-CSF (50 ng/ml) for 6 days (M0 macrophage) and then incubation for 24 h with GM-CSF, IL4, IL10, TGF beta at 20 ng/ml. M1 macrophage phenotype was obtained by adding GM-CSF(50 ng/ml) for 6 days (M0 macrophage) and then incubation for 24 h with GM-CSF (20 ng/ml), IFN gamma (20 ng/ml), LPS (50 ng/ml).

MTT Assays

For MTT assays, freshly lymphocytes were plated at a density of 1×10⁵ cells/in 96 well plates. The cells were cultured at 37° C. in 5% CO₂ for 48 h in RPMI medium containing 2% of inactivated fetal bovine serum added with antibodies (anti-CD9P-1 clone 9bF4 and its isotype control) at different concentrations, before the MTT staining and measurements according to the manufacturer's instructions. OD values at 490 nm were recorded and used to calculate the cell proliferation.

Co-Culture of Cancer Cells with PBMCs

Human non-small-cell lung carcinoma NCI-H460 cells (ATCC® HTB-177TH) were pre-seeded at a density of 2.5×10⁵ cells in a 6-well plate overnight in RPMI1640 media supplemented with 10% FBS at 37° C. The isolated PBMCs (effector cells, E) were added onto cancer cells (target cells, T) at a ratio of E:T=20:1 and cultured at 37° C., 5% CO₂ during 18H. Then, co-cultures were incubated for all treatment conditions for 48 h with RPMI medium containing 2% of fetal bovine serum. In some experiments, detached cells (lymphocytes) were removed to co-cultivate cancer cells with only adherent monocytes.

Enzyme-Linked Immunosorbent Assay (ELISA)

Lymphocytes were plated at a density of 5×10⁶ cells in six-well plates. Monocyte and M2 macrophage assays were realized from PBMCs initially plated at a density of 10×10⁶ cells in six-well plates. All cell types were incubated for 48 h with culture medium containing 2% of inactivated fetal bovine serum with anti-CD9P-1 antibody clone 9bF4 and isotype control. After incubation, supernatants were collected and stored at −20° C. Cytokines (TNF alpha, IFN gamma) were quantified with ELISA kits according to manufacturer's instructions (Peprotech, Neuilly-sur-Seine, France).

Western Blotting

After 48 h of incubation, cells were directly lysed in six-well plates in the lysis buffer. Proteins were extracted and quantified by Bradford assay. Cell lysates containing the same quantity of protein were resolved by SDS-PAGE with lysis buffer, resolved in SDS-PAGE. Proteins were transferred to PVDF membrane (Biorad system, Marnes-la-Coquette, France) and blotted with primary antibodies, anti-CD9P-1 (229t) described by Guilmain et al., (2011), anti-CD163, anti-CD80 (Abeam, Paris, France), anti-stabilin-1 (Millipore, Saint Quentin en Yvelines, France), anti-cleaved caspase-3, anti-TRAF-2, anti-Granzyme B and anti-GAPDH (used for normalization) from Cell Signaling Technology (Ozyme, Montigny-le-Bretonneux, France). Horse anti-mouse or goat anti-rabbit IgG antibodies coupled to horseradish peroxidase specific were used as secondary antibodies. Immunodetection was achieved with a chemiluminescence reagent (Thermofisher Scientific, Cergy Pontoise, France).

Immunohistochemistry on Tumor Sections

The immunostaining of human tumor sections was performed as follows. Deparaffination, rehydration, epitopes retrieval (CD9P-1, Stablilin-1: pH6, CD163: pH9) and the immunohistochemistry staining were fully automated using the Leica BOND-S MAX system according to manufacturer's instructions. The slides were developed using the Bond™ Polymer Refine Detection kit (Leica, Paris, France) and scanned with the Lamina scanner (Perkin Elmer, Villebon-sur-Yvette, France). The 4 μm sections were immunolabelled with the anti-CD9P-1 (clone 229t), anti-CD163 and anti-Stabilin-1 antibodies.

CD9P-1 Sandwich ELISA

A sandwich ELISA was developed to quantify CD9P-1 expression in cell lysates and tumor tissues. Monoclonal mouse anti-CD9P-1 mAb (clone 9bF4) was used as capture antibody and monoclonal mouse anti-CD9P-1 mAb (clone 229t) conjugated with biotin using EZ-link NHS-LC biotinylation kit (Thermofisher Scientific, Cergy Pontoise, France), as detection antibody. 96-well PVC microtiter plates were coated with the capture antibody at 10 μg/ml (200 μl) in PBS, covered with an adhesive plastic and incubated overnight at 4° C. After two washes with 0.05% tween 20 in PBS (diluent reagent), the remaining protein-binding sites in the coated wells were blocked with 1% BSA in PBS for 1 hour at room temperature. After two washes with 0.05% tween 20 in PBS, 100 μl of appropriately diluted samples was added to each well. Plates were incubated for 2 hours at room temperature. After 3 washes, 229t anti-CD9P-1 mAb was added at 2 μg/ml in 1% BSA in PBS+tween 0.05% and plates were incubated for 2 hours at room temperature. After 3 washes, Streptavidin-HRP diluted 1/8000 in diluent reagent was added for 30 min at room temperature in dark condition. The signal was revealed by addition of Ultra-TMB substrate after incubation for 30 min at room temperature, followed by 2 M H2SO4 (v/v) (Thermofisher Scientific, Cergy Pontoise, France). Absorbance of each well was measured at 450 nm with a microplate reader equipped with KC4 software (BioTek Instruments, Colmar, France). The CD9P-1 amount was determined by comparison to a standard curve using a known amount of the recombinant protein produced in CHO system. Cell lysates were obtained from the cell lines Lewis (LL/2), NCH-H460, RT-112, Umuc-3, NCI-H69 FaDu, A649, A673, U937, Jurkat, THP-1, (ATCC), RAW264.7 (Sigma), RH-30 (DSMZ). Tumor Lysates were obtained from tumor xenografts in mice model.

CD9P-1 Internalization and Degradation

K562 cells were incubated in RPMI1640 media supplemented with 2% FBS at 37° C. overnight and then seeded at a density of 1×106 cells in a 6-well plate in fresh media with 10 per ml of the Mouse IgG 9bF4, or an IgG isotype control for different times. After incubation, cells were centrifugated at 220 g, 10 min, washed twice with PBS and lysed in 1% Triton X100 CST cell lysis buffer containing protease inhibitors. The analysis of CD9P1 expression was then assessed by Western blot with anti-CD9P-1 mAb.

NCI-H460 cells were grown in RPMI containing 10% FCS in T-175 Flasks at 37° C. and 5% CO2 humidified atmosphere. When cells reached a confluence of 80-90%, the medium was replaced with serum-free medium for 4 hours and then change to complete medium. Cells were then treated with 9bF4 mAb at 10 μg/ml for 0.5, 2 and 18 h or a control IgG1 for 18 h. The subcellular proteome fractions were prepared using a ProteoExtract Subcellular Proteome Extraction kit (EMD Millipore, Saint Quentin en Yvelines, France) according to the manufacturer's instructions. The subcellular proteome fractions were subjected to western blot analysis with anti-CD9P-1 and anti-TRAF-2 mAbs.

CD9P-1 Internalization as Assessed By Flow Cytometry

K562 cells were deprived of serum for 5 hours, seeded at a density of 1.5×10⁶ cells in a 6-well plate in RPMI 10% FBS in presence of 10 μg of anti-CD9P-1 antibodies for 30 min at 4° C. and then at 37° C. at the indicated times. Internalization was stopped at 4° C. Cells were centrifuged at 220 g, 5 min, at 4° C., washed twice with cold PBS and specifically bound antibodies were stained with a PE F(ab′)2-Goat anti-Mouse IgG secondary antibody (Thermofisher-Scientific, Courtaboeuf, France), and analyzed with a cytometer FC500 (Beckman Coulter, Villepinte, France) to measure the antibodies internalized by K562 cells. The data were analyzed with CXP software (Beckman Coulter). Another isolated anti-CD9P1 mAb (clone 13aA6) was used as a positive control of CD9P-1 recognition on cell surface but negative control of internalization.

Epitope mapping of 9bF4 mAb

The epitope recognized on human CD9P1 antigen by the mouse anti-CD9P1 (9bF4mAb) was determined by using the high-resolution method developed by CovalX (WO 2017/121771). 9bF4 mAb was complexed with a soluble preparation of human CD9P1 extracellular domain (Strep-CD9P1-ECD). The protein complex was incubated with deuterated cross-linkers and subjected to multi-enzymatic proteolytic cleavage. After enrichment of the cross-linked peptides, the samples were analyzed by high resolution mass spectrometry (nLC-Q Exactive Orbitrap MS) and the data generated were analyzed using XQuest and Stavrox software.

In vivo Efficacy of Mouse Anti-hCDPI Antibody 9bF4 mAb

9bF4 mAb was evaluated as single agent in mouse xenograft model using K562 (human Chronic Myeloid Leukemia) and NCI-H460 (human Non-Small Cell Lung Carcinoma). Nude mice (n=4 per group) were engrafted with K562 or NCI-H460 cells (5 million cells injected subcutaneously). The antibody therapy was started fourteen days after the cell transplant, once the tumor volume was about 100 mm³. The anti-CD9P1 antibody 9bF4 was injected alone at the dose of 10 mg/kg/dose by intraperitoneal injection, 2 times a week (for 2 weeks).

Results

Characterization of 9bF4 and 10bB1 mAbs Specificity By Flow Cytometry Using CD9P1-Expressing or -Non-Expressing Cancer Cell Lines:

Firstly, we aimed at characterizing the specificity of the 9bF4 and 10bB1 antibodies of the invention for the human CD9P-1 protein. In this regard, we first analyzed CD9P-1 expression in cell lysates from two cancer cell lines (Jurkat and K562) using QPCR (FIG. 1) and western Blot analysis with the CD9P-1-targeting antibody (FIG. 2).

The following primer pairs were used for QPCR:

| (SEQ ID NO: 37) | |
|---|---|
| hCD9P1-F | 5′-CAGGAGCTGGCACAAAGTG-3′ |
| and | |
| (SEQ ID NO: 38) | |
| hCD9P1-R | 5′-CCTTGGAAGCATTCAGGTACA-3′; |
| (SEQ ID NO: 39) | |
| hGAPDH-F | 5′-AGCTCACTGGCATGGCCTTC-3′ |
| and | |
| (SEQ ID NO: 40) | |
| hGAPDH-R | 5′-GAGGTCCACCACCCTGTTGC-3′. |

We then tested hybridomas supernatants by an indirect flow cytometry method using a PE-conjugated goat anti-mouse antibody to isolate hybridomas producing anti-CD9P-1 antibodies. After cloning of positive hybridomas by ELISA, supernatants from the clones 9bF4 and 10Bb1 showed a high staining of the K562 cell line and were negative on Jurkat cells (FIG. 3). After purification, 9bF4 and 10bB1 mAbs showed the same specificity with high staining of K562 and negative labelling on Jurkat cell lines (FIG. 4).

Characterization of 9bF4 and 10bB1 mAbs Specificity by Immuno-Precipitation:

Then, we investigated CD9P-1 expression in CD9P-1-transfected (or GFP/non-transfected) CT26 cells by QPCR (FIG. 5) and western Blot with the anti-CD9P-1 mAb (FIG. 6A). The following primer pairs were used for QPCR:

| hCD9P1-F and hCD9P1-R; | |
|---|---|
| (SEQ ID NO: 41) | |
| mGAPDH-F | 5′-GGCCTTGACTGTGCCGTTGAATTT-3; |
| and | |
| (SEQ ID NO: 42) | |
| mGAPDH-R | 5′-GGCCTTGACTGTGCCGTTGAATTT-3′. |

We noticed that CD9P-1 was overexpressed by CD9P-1-transfected CT26 cells but weakly expressed by non-transfected CT26 cells. Cell lysates of CD9P-1-transfected (or non-transfected) CT26 cells were next immunoprecipitated with the 9bF4 mAb of the invention before being immunoblotted for CD9P-1 using the CD9P-1-targeting mAb. The 135 kDa form of CD9P-1 immunoprecipitated by the 9bF4 mAb was detected by immunoblot with the CD9P-1 mAb (FIG. 6B). Thereafter, K562 cell lysates were immunoprecipitated with 9bF4 or 10bB1 hybridoma supernatants; and H460 cell lysates were immunoprecipitated with 9bF4 and 10bB1 purified mAbs. Samples were separated by SDS-PAGE and immunoblotted with the anti-CD9P-1 mAb. The 135 kDa form of CD9P-1 was finally successfully immunoprecipitated by 9bF4 and 10bB1 mAbs in the two CD9P1-expressing human cancer cell lines (FIG. 7A). These data thus demonstrate the specificity of 9bF4/10bB1 mAbs for the human CD9P-1 protein.

The CD9P-1-Targeting Antibodies of the Invention (9bF4 and 10bB1) have the Unique Characteristic of Being Rapidly and Efficiently Internalized In the Cytoplasm of CD9P-1-Expressing Non-Adherent Cancer Cells:

Thereafter, to investigate whether the 9bF4 and 10bB1 mAbs are internalized in the cytoplasm of CD9P-1-expressing cancer cells upon binding to the cell surface, K562 leukaemia cells were incubated with 10 μg/m1 of 9bF4 and 10bB1 mAbs at 4° C. for 30 min. Of note, an antibody binding to CD9P-1 but which cannot be uptaked within cancer cells was used as a control of internalization (clone 13aA6). After washing, the cells were incubated at 37° C. to allow for internalization. Then, after several time periods (0, 15, 30 min, and lh, 2 h and 18 h), cell samples were collected and stained with a PE-labeled secondary antibody at 4° C. Finally, the corresponding cell surface PE signals were analysed with a flow cytometer. The results show that K562 cells efficiently and rapidly internalized 9bF4 and 10bB11 antibodies compared to control (13aA6 mAb) (FIG. 7B).

The Antibody of the Invention (9bF4 or 10bB1) Induces CD9P-1 Degradation In CD9P-1-Expressing Non-Adherent Cancer Cells:

Lastly, to determine if 9bF4 and 10 bB1 mAb binding to CD9P1 regulates its expression level in cancer cells, non-adherent K562 cells were incubated with the Mouse IgG 9bF4 or IgG 10bB1, or an IgG isotype control for different times. After incubation, cells were lysed and CD9P1 expression was analyzed by Western blot. Interestingly, we noticed a complete depletion of CD9P-1 protein expression induced by 9bF4 mAb from 5 hours of incubation. No CD9P-1 expression was detected until 72 h of incubation with 9bF4 mAb (FIG. 7C). Furthermore, we observed a degradation of CD9P1 with the two antibodies in comparison with control condition (C) and the kinetic of degradation induced by 9bF4 mAb or 10bB1 mAb is very similar with a strong depletion of CD9P-1 protein expression after 2 h of incubation (FIG. 7E).

The antibody of the invention (9bF4) triggers CD9P-1 internalization and degradation in CD9P-1-expressing adherent cancer cells:

Eventually, to further demonstrate that the 9bF4 mAb induces CD9P-1 internalization and degradation in CD9P-1-expressing cancer cells, adherent NCI-H460 cells were incubated with 9bF4 mAb for 0.5, 2 and 18 h or with a control IgG1 for 18 h. The subcellular proteome fractions were then subjected to western blot analysis. We found that 9bF4 mAb drastically and rapidly decreased (starting as soon as 0.5 h after incubation) CD9P-1 expression in membrane and cytosolic fractions of NCI-H460 cells, thus indicating that 9bF4 induces a rapid internalization and degradation of CD9P-1 in these cells (FIG. 7D). These findings are further supported by FIG. 7B which demonstrates the rapid internalization of 9bF4 and 10bB1 (as soon as 1 h after incubation) by CD9P-1-expressing cancer cells. Moreover, considering that TRAF-2 has an anti-apoptotic activity and its depletion leads to tumor growth suppression, we further examined whether 9bF4 mAb may downregulate TRAF-2 through its interaction with CD9P1 in cancer cells. For that, we performed immunoblot analysis of TRAF-2 expression in aforementioned cancer cells, incubated or not with 9bF4 at different times after subcellular fractionation. We observed that 9bF4 drastically deplete expression of TRAF-2 in cytosolic fraction, thus pointing out that 9bF4 could also induce apoptosis in CD9P1-expressing cancer cell through TRAF-2 degradation and TRAF-2/TNFR signaling (FIG. 7D).

The Antibody of the Invention (9bF4) Stimulates The Production of a Pro-Inflammatory Cytokine (TNF-Alpha) In Human Monocytes and M2 Macrophages:

To investigate whether the CD9P-1-targeting antibody of the invention have an immunostimulatory effect on human innate immune cells, we treated monocytes and M2 macrophages with different concentrations (10-20 μg per ml) of the herein mentioned CD9P-1-targeting antibody (Mouse IgG 9bF4), or an IgG isotype control, and further performed an ELISA assay on TNF-alpha. Interestingly, we found that the IgG 9bF4 antibody induces a strong release of TNF-alpha by human monocytes (FIG. 8A) and M2 macrophages (FIG. 8B) which indicates that 9bF4 may induce both the cancer cell apoptosis through macrophages in tumor environment and M1 macrophage polarization which is characterized by TNF-alpha production.

The Antibody of the Invention (9bF4) Induces the Production of Pro-Inflammatory Cytokines (TNF-Alpha and IFN-Gamma) In Human Lymphocytes:

Then, to determine whether the antibody of the invention have an immunostimulatory effect on human adaptive immune cells, we treated lymphocytes with different concentrations (5-20 μg per ml) of the CD9P-1-targeting antibody (Mouse IgG 9bF4), or an IgG isotype control, and further performed ELISA assays on TNF-alpha and IFN-gamma. Noteworthy, we found that the antibody of the invention induces a potent secretion of TNF-alpha (FIG. 9A) and IFN-gamma (FIG. 9B) by human lymphocytes.

The Antibody of the Invention (9bF4) Triggers the Proliferation of Human Lymphocytes:

To further investigate the immunostimulatory potential of the anti-CD9P-1 antibody on human adaptive immune cells, we treated lymphocytes with different concentrations (5-40 μg per ml) of the 9bF4 antibody, or an IgG isotype control, and further performed a colorimetric MTT assay for measuring cell proliferation. Strikingly, we found that the antibody of the invention strongly stimulates human lymphocytes proliferation (FIG. 10).

The Antibody of the Invention (9bF4) Elicits Human M2 to M1 Macrophage Repolarization Through Stabilin-1 Pathway:

Besides, given that M2 macrophages have been shown to promote tumors angiogenesis and metastasis; we asked if the antibody of the invention may trigger M2 to M1 macrophage polarity shift, thereby limiting the deleterious anti-inflammatory and protumor effects of M2 state macrophages. To do so, we performed immunoblot analysis of M1/M2 macrophage (CD80/CD163) and M2-polarized macrophage (Stabilin-1) molecular markers over M1 or M2 macrophage lysates. M2 macrophages were incubated for 48 h with a CD9P-1-targeting antibody (Mouse IgG 9bF4, 20 μg per ml) or its IgG isotype control. CD9P-1 mAb dropped CD163 expression (M2 macrophage) and induced CD80 overexpression (M1 macrophage), thus indicating that the antibody of the invention in fact promotes M2 to M1 macrophages repolarization (FIG. 11). Furthermore, 9bF4 mAb induced CD9P1 and Stabilin-1 simultaneous degradation, pointing out that 9bF4 mAb triggered an immunostimulatory response through the internalization and degradation of an CD9P1/stabilin-1 molecular complex (FIG. 11).

The Antibody of the Invention (9bF4) Induces the Production of Pro-Inflammatory Cytokines (TNF-Alpha and IFN-Gamma) In Human PBMC Co-Cultured with Cancer Cells:

Thereafter, to ensure that the previously identified immunostimulatory effects of the CD9P-1 antibody is further noticed in the presence of human metastatic cancer cells (NCI-H460); we treated human peripheral blood mononuclear cells (PBMCs) with different concentrations (10-20 μg per ml) of the Mouse IgG 9bF4, or an IgG isotype control, and further performed ELISA assays on TNF-alpha and IFN-gamma. Of note, we observed that the 9bF4-induced increased production of inflammatory cytokines is still obvious in the presence of NCI-H460 cancer cells (FIGS. 12A-B); and more interestingly, we found that the immunostimulatory effect of the 9bF4 antibody was potentiated for IFN-gamma in the presence of these tumor cells (FIG. 12B).

The Antibody of the Invention (9bF4) Triggers CD9P-1-Expressing Cancer Cell Apoptosis Through Monocyte and Lymphocyte Activation:

Finally, given the previously identified immunostimulatory effects of the 9bF4 antibody; we examined if the antibody of the invention may actually regulate human CD9P-1-expressing cancer cells apoptosis. Importantly, we found that the Mouse IgG 9bF4 antibody, but not the IgG isotype control, indeed induces increased NCI-H460 cells apoptosis when co-cultured with monocytes or monocytes/lymphocytes derived from PBMC, as revealed by immunoblotting for the apoptosis marker: cleaved-caspase 3. (FIG. 13). Interestingly, this increased cancer cell death is more significant when cancer cells are cultured with both monocytes and lymphocytes than with monocytes alone, thus showing an amplified apoptotic effect in presence of lymphocytes, i.e. presuming of a robust and synergistic innate and adaptive immune response As the antibody of the invention elicited lymphocyte proliferation, we investigated if it may also induce cytotoxic function of lymphocytes and we thus examined granzyme B expression levels in cytotoxic granules of human PBMC co-cultured with cancer cells. Surprisingly, we found that the antibody of the invention, but not the IgG isotype control, triggers a dramatic increase in granzyme B expression from lymphocytes, which is a predictive biomarker of immunotherapy response (FIG. 13).

The CD9P-1 and stabilin-1 proteins are over-expressed in human M2 state macrophages and coexpressed in human cancerous lung tissue:

Then, we evaluated the expression profile of CD9P-1 in various human macrophages by performing immunoblot assay on human M0 or M1/M2 macrophage cell lysates, using M2 (CD163) molecular marker. We noticed that CD9P-1 and Stabilin-1 are over-expressed in M2 macrophages compared to M0/M1 macrophages (FIG. 14A). Moreover, we found that CD9P-1 expression in M2 state macrophages correlates with stabilin-1 increased expression (FIG. 14A).

Further, we investigated the expression profile of CD9P-1 and stabilin-1 in human lung adenocarcinoma after chemotherapy with cisplatin and permetrexed (Alimta™), using immunohistochemical staining, with a M2 macrophage marker (CD163). Interestingly, we found that CD9P-1 and stabilin-1 proteins are coexpressed in an advanced case of human lung cancer after a first line treatment, and correlates with the M2 macrophage phenotype in tumor stroma (FIG. 14B). The results validate M2 macrophages in the tumor environment as a promising target for tumour therapy with 9bF4, even after a chemotherapy with cisplatin and permetrexed.

The Anti-CD9P-1 mAb (9bF4) of the Invention Induces Immune Response During the M1 Repolarization Through TRAF-2/TNFR Signaling:

TRAF-2 regulates inflammatory cytokine production in tumor-associated macrophages, facilitates tumor growth and, interestingly, it was suggested that loss of TRAF-2 may promote the M1-like anti-tumor function of macrophages. Thus, given that 9bF4 regulates TNF-alpha production in human immune cells and that TRAF-2 is an adaptor protein that is well-known for transducing signals following ligation of certain cytokine receptors including those binding TNF, we further examined whether the antibody of the invention (9bF4) may modulate TRAF-2 pathway in human macrophages. To do so, we performed immunoblot analysis of TRAF-2 expression in human monocytes and M1 vs M2 macrophages, incubated or not with 9bF4 (or an IgG isotype control) for 48 h. We noticed that 9bF4 decreased TRAF-2 expression in M2 macrophages, thus indicating that the immune response induced by 9bF4 during M1 macrophage repolarization in fact occurs through TRAF-2/TNFR signalling (FIG. 15).

The Antibody of the Invention (9bF4) Inhibits In Vivo Tumour Growth:

As shown in FIG. 16, the anti-CD9P1 antibody 9bF4 mAb significantly delays the growth of tumor in treated-mice when compared to the control group of animals injected with the vehicle (p<0.05, Student's t-test). Thus, these results show an anti-tumoral activity of the mouse anti-hCD9P1 (9bF4) in athymic mice xenografted with cancer cell expressing human CD9P-1.

The Antibody of the Invention (9bF4) Recognises a Conformational Epitope on CD9P-1:

The chemical cross-linking analysis showed that the epitope on Strep-CD9P1-ECD recognized by 9bF4 mAb is discontinuous (conformational epitope). 9bF4 mAb binds to one or more amino acids within amino acid residues 210-240, 430-450, 480-510 in Strep-CD9P1-ECD (corresponding respectively to amino acid residues 202-232, 422-442 and 472-502 in CD9P-1) including the following amino acids: 219, 222, 223, 232, 433, 444, 480, 482, 486, 505 and 509 (corresponding respectively to amino acid residues 211, 214, 215, 224, 425, 436, 472, 474, 478, 497 and 501 in CD9P-1). As 10bB1 shares identical CDR sequences with 9bF4 mAb, it was deduced that 10bB1 recognizes the same epitope as 9bF4 mAb.

Determination of the affinity of the antibody of the invention (9bF4 or 10bB1) for human CD9P-1:

9bF4 mAb and 10bB lmAb binding to Strep-CD9P1-ECD and their affinity to Strep-CD9P1-ECD were assessed using surface plasmon resonance (SPR). Recombinant Strep-CD9P1-ECD was coated on sensor chip and several concentrations of the antibodies were flowed over the chip to obtain binding and dissociation kinetics. The results are shown in Table 1 below.

TABLE 1
Kinetics parameters and affinity of antibody/antigen interaction

| Antibody | ka (1/Ms) | kd (1/s) | KD (M) |
|---|---|---|---|
| 9bF4 | 5.41 × 10⁴ | 4.91 × 10⁻³ | 9.09 × 10⁻⁸ |
| 10bB1 | 1.32 × 10⁴ | 1.18 × 10⁻² | 8.99 × 10⁻⁷ |

1-21. (canceled)

22. An isolated protein that inhibits the CD9P-1 pathway.

23. The isolated protein according to claim 22, wherein said protein inhibits the CD9P-1/stabilin-1 pathway and/or the CD9P-1/TRAF-2 pathway.

24. The isolated protein according to claim 22, wherein said protein induces an internalization and/or degradation of CD9P-1.

25. The isolated protein according to claim 22, wherein said protein induces an internalization and/or degradation of Stabilin-1 and/or degradation of TRAF-2.

26. The isolated protein according to claim 22, wherein said protein binds to CD9P-1.

27. The isolated protein according to claim 26, wherein said protein is an antibody molecule selected from the group consisting of a whole antibody, a humanized antibody, a single chain antibody, a dimeric single chain antibody, a Fv, a Fab, a F(ab)′2, a defucosylated antibody, a bi-specific antibody, a diabody, a triabody, a tetrabody, an antibody fragment selected from the group consisting of a unibody, a domain antibody, and a nanobody or an antibody mimetic selected from the group consisting of an affibody, an affilin, an affitin, an adnectin, an atrimer, an evasin, a DARPin, an anticalin, an avimer, a fynomer, a versabody, and a duocalin.

28. The isolated protein according to claim 26, wherein said protein binds to a conformational epitope comprising:
at least one amino acid residue from amino acid residues 202 to 232 in human CD9P-1 (SEQ ID NO:34), or from a sequence sharing at least 60%, 70%, 75%, 80%, 90%, 95%, 96%, 97%, 98%, 99% of identity over amino acid residues 202 to 232 of human CD9P-1 (SEQ ID NO:34), and
at least one amino acid residue from amino acid residues 422 to 442 in human CD9P-1 (SEQ ID NO:34), or from a sequence sharing at least 60%, 70%, 75%, 80%, 90%, 95%, 96%, 97%, 98%, 99% of identity over amino acid residues 422 to 442 of human CD9P-1 (SEQ ID NO:34), and
at least one amino acid residue from amino acid residues 472 to 502 in human CD9P-1 (SEQ ID NO:34), or from a sequence sharing at least 60%, 70%, 75%, 80%, 90%, 95%, 96%, 97%, 98%, 99% of identity over amino acid residues 472 to 502 of human CD9P-1 (SEQ ID NO:34).

29. The isolated protein according to claim 26, being an antibody molecule or an antibody fragment, wherein the variable region of the heavy chain comprises at least one of the following CDRs:

| | | |
|---|---|---|
| VH-CDR1: | GYTFTSYW; | (SEQ ID NO: 1) |
| VH-CDR2: | IFPGTGTT; | (SEQ ID NO: 2) |
| and | | |
| VH-CDR3: | SRDFDV. | (SEQ ID NO: 3) |

or any CDR having an amino acid sequence that shares at least 60% of identity with SEQ ID NO:1-3, and/or
wherein the variable region of the light chain comprises at least one of the following CDRs:

| | | |
|---|---|---|
| VL-CDR1: | QSLLDIDGKTY; | (SEQ ID NO: 4) |
| VL-CDR2: | LVS; | |
| and | | |
| VL-CDR3: | WQGTHLPRT, | (SEQ ID NO: 5) |

or any CDR having an amino acid sequence that shares at least 60% of identity with SEQ ID NO:4, LVS, and SEQ ID NO:5.

30. The isolated protein according to claim 26, being an antibody molecule or an antibody fragment, wherein the variable region of the heavy chain comprises at least one of the following CDRs:

| | | |
|---|---|---|
| VH-CDR1: | GYTFTSYW; | (SEQ ID NO: 1) |
| VH-CDR2: | IFPGTGTT; | (SEQ ID NO: 2) |
| and | | |
| VH-CDR3: | SRDFDV, | (SEQ ID NO: 3) |

or any CDR having an amino acid sequence that shares at least 60% of identity with SEQ ID NO:1-3,
and the variable region of the light chain comprises at least one of the following CDRs

| | | |
|---|---|---|
| VL-CDR1: | QSLLDIDGKTY; | (SEQ ID NO: 4) |
| VL-CDR2: | LVS; | |
| and | | |
| VL-CDR3: | WQGTHLPRT, | (SEQ ID NO: 5) |

or any CDR having an amino acid sequence that shares at least 60% of identity with SEQ ID NO:4, LVS, and SEQ ID NO:5.

31. The isolated protein according to claim 26, being an antibody molecule or an antibody fragment, wherein the variable region of the heavy chain comprises the following CDRs:GYTFTSYW (SEQ ID NO:1), IFPGTGTT (SEQ ID NO:2) and SRDFDV (SEQ ID NO:3) and the variable region of the light chain comprises the following CDRs:QSLLDIDGKTY (SEQ ID NO:4), LVS, and WQGTHLPRT (SEQ ID NO:5) or any CDR having an amino acid sequence that shares at least 60% of identity with said SEQ ID NO:1-5 and LVS.

32. The isolated protein according to claim 26, being an antibody molecule or an antibody fragment, wherein the amino acid sequence of the heavy chain variable region is SEQ ID NO:6 wherein X₁ is Q or R, X₂ is R or G, X₃ is T or A, X₄ is S or T and the amino acid sequence of the light variable region is SEQ ID NO:7 whereinX₅ is P or L, X₆ is S or F, and X₇ is S or absent, or any sequence having an amino acid sequence that shares at least 60% of identity with said SEQ ID NO:6-7.

33. The isolated protein according to claim 32, wherein
in SEQ ID NO:6:
X, is R, X₂ is R, X₃ is T and X₄ is T (SEQ ID NO:8), or
X₁ is Q, X₂ is R, X₃ is T and X₄ is T (SEQ ID NO:11) and/or
in SEQ ID NO:7:
X₅ is L, X₆ is S and X₇ is S (SEQ ID NO:24).

34. A method for treating cancer and/or tumor in a subject in need thereof, comprising administering to the subject the isolated protein according to claim 22.

35. The method according to claim 34, wherein the method is for inducing apoptosis of cancer cells in the subject.

36. The method according to claim 34, wherein the method is for inducing M2 macrophages repolarization in M1 macrophages in the subject.

37. The method according to claim 34, wherein the method is for inducing an immune response and/or an inflammatory response in the subject.

38. A method for detecting CD9P-1 in a sample, comprising the use of the isolated protein according to claim 22.

39. The method according to claim 38, wherein the method is an in vitro diagnostic or prognostic method.

40. The method according to claim 38, for determining the presence of the 135 kDa glycosylated transmembrane form of CD9P-1 in a biological sample.

41. The method according to claim 38, wherein the method comprises the use of an assay being a sandwich ELISA using as coating antibody an antibody wherein the variable region of the heavy chain comprises the following CDRs:GYTFTSYW (SEQ ID NO:1), IFPGTGTT (SEQ ID NO:2) and SRDFDV (SEQ ID NO:3) and the variable region of the light chain comprises the following CDRs:QSLLDIDGKTY (SEQ ID NO:4), LVS and WQGTHLPRT (SEQ ID NO:5) or any CDR having an amino acid sequence that shares at least 60% of identity with said SEQ ID NO:1-5 and LVS.
